(19) United States
(12) Patent Application Publication
(10) Pub. No.: US 2006/0223053 A1
(43) Pub. Date: Oct. 5, 2006

(54) Direct measurement of sorption on three-dimensional surfaces such as resins, membranes or other preformed materials using lateral dispersion to estimate rapid sorption kinetics or high binding capacities

(76) Inventor: D. Keith Roper, Salt Lake City, UT (US)

Correspondence Address: TRASK BRITT, P.O. BOX 2550, SALT LAKE CITY, UT, 84110, US

(21) Appl. No.: 11/271,141

(22) Filed: Nov. 9, 2005

Related U.S. Application Data
(60) Provisional application No. 60/626,566, filed on Nov. 9, 2004.

(51) Int. Cl. C12Q 1/70; G01N 33/53; C12M 1/34

(52) U.S. Cl. 435/5; 435/7.1; 435/287.2

(57) ABSTRACT

Described are methods allowing measurement of adsorption and desorption of analytes with membranes or resins directly using surface plasmon resonance (SPR). Also described are methods for assembling intact resins or membranes on SPR surfaces. Such methods provide estimates of mass-action ion-exchange adsorption and desorption rates accounting for steric (σ) and characteristic charge (ν) effects. The methods further permit accurate estimation of rate constants for uniform adsorption of a homogeneous analyte solution on homogeneous adsorptive sites distributed heterogeneously in space, relative to the planar boundary. Solutions are obtained for locally porous media and solid spheres. The methods are extendible to other media and heterogeneous adsorptive sites. The methods further provide for enhancement of lateral mass transport in such optical measurement instruments through radial hydrodynamic diffusion (radial dispersion) by, for example, incorporating porous media in flow cells of detection devices such as, but not limited to, SPR or TIRF instruments.

### CROSS-REFERENCE TO RELATED APPLICATION

This application claims priority under 35 U.S.C. 199(e) to U.S. Provisional Application 60/626,566, filed Nov. 9, 2004, the contents of the entirety of which is incorporated herein by reference.

### TECHNICAL FIELD

The invention relates to the field of biotechnology and optical multi-analyte biosensor technology, more particularly to the use of optical multi-analyte biosensor technology employing the principal of internal reflection of polarized light for use in biological, biochemical and chemical analysis and in particular for detecting interactions or binding events between biological molecules, such as adsorption of viral particles to three-dimensional surfaces allowing measurement of adsorption and desorption of analyte(s) to intact membranes or resins directly. The detection method used in the biosensor system may be based on the evanescent wave phenomenon at total internal reflection, such as surface plasmon resonance (SPR), critical angle refractometry, total internal reflection fluorescence (TIRF), total internal reflection phosphorescence, total internal reflection light scattering, optical waveguide fluorescence and evanescent wave ellipsometry.

### BACKGROUND OF THE INVENTION

Surface plasmon resonance (“SPR”) has been widely used to analyze biospecific interactions like antibody-antigen or receptor-ligand events, including evaluating effects of kinetics, binding site and concentration [6]. In a typical SPR experiment, one particular type of biomolecule (such as an antibody), referred to as the “ligand,” is immobilized on the surface of a sensor chip, and another type of biomolecule (such as an antigen), the “analyte,” is propelled over the surface of the sensor chip in a fluid discharge at a constant rate in a constant concentration.

As used herein, the term “analyte” refers to a substance that is undergoing analysis or is being measured. The entire analysis is conducted under buffer conditions biochemically suitable to the analyte. As the ligand and analyte interact, the SPR apparatus, consisting of various lenses and detectors, is used to measure changes in refractive index on the surface of the sensor chip. Binding of the analyte by the ligand results in an increase in refractive index which is monitored in real time by a change in the resonance angle as measured by SPR. The data take the form of a sensorgram which plots the signal in resonance units (RU) as a function of time. Such instruments are well characterized in the literature and the subject of numerous patents, such as WO 90/05303, WO 90/05305, and U.S. Pat. No. 5,313,264, the contents of all of which are incorporated herein by this reference.

Without applying secondary reagents for signal enhancement, SPR techniques can detect adsorption of protein solutions as dilute as 7-10 nM. SPR decreases the intensity of light reflected at a specific angle from a conducting gold film adjacent to a dielectric medium (glass and sample). A schematic of surface plasmon resonance measurement is shown in FIG. 2. At the angle of minimum reflectance, incident light photons excite delocalized metal electrons, causing them to oscillate collectively and dissipate light energy. The minimum angle varies with the sample refractive index close to the glass surface. Molecules in the sample that bind to the sensor surface change the local refractive index, thereby shifting the angle of minimum reflectance and producing a measurable SPR response.

Many diseases such as cancer, diabetes, hemophilia, cystic fibrosis, heart disease and musculoskeletal disorders have an underlying genetic basis. Sequencing the human genome has improved the ability to identify alterations in relevant DNA sequences that could be remedied to correct or prevent gene-related disorders. One treatment approach is gene therapy: inserting correct copies of the altered gene into non-germline cells using nanometer-sized viral or synthetic liposome vectors as delivery vehicles. There are about nine hundred clinical trials currently in progress to administer gene therapy [1]. Over 60% of these trials target cancer cells, about 10% treat monogenic diseases like hemophilia or cystic fibrosis, and about 6% combat infectious diseases like HIV. About 75% of gene vectors used clinically are virus derivatives. Most clinical studies use retrovirus (28%), followed by adenovirus (26%).

Adenovirus Type 5 (Ad5) is a non-enveloped double-stranded DNA viral vector [2]. Its structure, physical properties and protein composition are shown in FIG. 1. The 36-kilobasepair genome of Ad5 is encapsidated in a rigid, 80-nm icosahedron composed of 12 different proteins [3]. Within the Ad5 vector genome, the E1 typically replication region is replaced by the selected therapeutic transgene or nucleic acid sequence of interest. Ad5 capsid contains twelve 32-nm-long fibers protruding from 12 penton base vertices that mediate electrostatic interactions with cellular receptors. Ad5 attaches to cells by binding its 32-nm fiber to the a2 domain of major histocompatibility complex class I (HLA MHC), or its fiber knob to coxsackievirus-adenovirus receptor (CAR). The penton base then interacts with αᵥβ₃ and αᵥβ₅ integrin receptors to internalize Ad5 by receptor-mediated endocytosis into clathrin-coated pits. Genetic DNA within the protective Ad5 protein sheath is then transferred to the nucleus.

In order to conduct gene therapy clinical trials, recombinant viral particles are propagated. This propagation is typically achieved by infecting host human mammalian cell lines with viral particles, allowing the virus to multiply, then harvesting the cells to isolate the viral particles. The viral particles are then purified for clinical use. Recovery of adenoviral gene vectors from replication in host mammalian cell lines typically involves cell lysis by freeze-thaw; microfiltration or precipitation of debris; anion-exchange chromatography to remove cell protein, DNA and defective virus; and ultrafiltration for buffer exchange. High-resolution Ad5 adsorption steps are vital, since the U.S. Food and Drug Administration, Center for Biologics Evaluation and Research, which regulates biological products administered in the U.S., recommends <100 picograms of residual DNA per dose from mammalian-cell products and <100 virions per infectious unit [4].

Currently, Ad5 chromatography is limited by the scarcity of published research on Ad5 adsorption, the cost of resins for Ad5 and the fact that existing resins are optimized for small synthetics (≦10⁴ Da) or recombinant proteins (≦10⁶ Da). Consider two examples: Spherodex® LS resin (Ciphergen®, a non-compressible silicadextran composite sorbent) has 100-nm pores that exclude particles greater than 10⁷ Da including adenovirus (1.65×10⁸ Da). Resource™ Q resin (Amersham Biosciences, monodisperse polystyrene/divinyl benzene beads) has a detection limit for adenovirus that exceeds 1×10⁸ particles per milliliter [5]. Furthermore, its prohibitive cost dominates manufacturing expenses.

Estimates for on and off rate constants and dissociation constants for interaction between Ad5 fiber knob and CAR domains are reported in the literature using SPR.[8,9] Association equilibrium constants for protein interaction with affinity ligands measured by SPR were reported to be comparable to those measured by from chromatographic breakthrough curves using Scatchard analysis [10]. Significantly, SPR measurements of equilibrium constants were 15-fold faster and used 130-fold less protein than chromatographic breakthrough-curve measures of the same equilibrium parameters.

However, while the SPR technology offers new vistas of possibilities in bioscience research, it is still a limited technique. Applying existing SPR measurements of biomolecule-polymer surface interactions to rationally design three-dimensional surfaces is very limited because current SPR sensor chip surfaces are based only on one-dimensional ideal surfaces and available software packages used to fit binding rate constants to SPR sensorgrams are currently based only on a simple bimolecular interaction between an analyte and a ligand which neglects steric or characteristic charge effects in mass-action ion exchange applications.

### SUMMARY OF THE INVENTION

The present invention may be used to identify optimum adsorbate physicochemistry and binding conditions for biomolecules on synthetic or biological three-dimensional surfaces, allow screening of candidate surfaces and providing insight into three-dimensional surface structures. This method of the invention allows improved efficiencies in isolation and purification of viral particle stocks for use in various applications, such as, but not limited to, gene therapy. To enable more efficient isolation and purification of viral particles, the interaction of viral particles with materials typically commercially available for such isolation methods, such as diethylaminoethyl (DEAE) ion exchange resins, must be characterized and studied.

Herein we disclose the effects of diffusive mass transport and ionic strength on nonspecific electrostatic interactions between DEAE modified surfaces and cytochrome c and Ad5. This invention is the first to overcome many technical limitations (such as lateral mass transport limitations), allowing measurement of sorption rate constants for nonspecific electrostatic interactions between large proteins or virus particles and a synthetic ion-exchange surface using SPR. Disclosed herein for the first time is a method for detecting and measuring binding kinetics of whole adenoviral particles, rather than capsid or fiber proteins. Further disclosed detecting and measuring binding kinetics of an analyte on a three-dimensional surface.

A gold SPR surface was derivatized with a 11-mercaptoundecanoic acid (MUDA) self assembled monolayer (SAM) which was then substituted with commercially available DEAE resin. Parameter estimates were obtained at analyte concentrations near the limit of SPR detectability by fitting sensorgrams to a two-compartment mass-transport limited reaction model. Estimates for adsorption, desorption and dissociation of cytochrome c on DEAE-SAM were obtained at protein levels as low as 8.1 nM. It is disclosed herein that cytochrome c adsorption rate measured at negligible NaCl content (6.9±0.053×10⁴ M⁻¹s⁻¹) is comparable to the rate of mass transport to the surface by diffusion. Estimates were obtained for Ad5-DEAE sorption using Ad5 solutions as dilute as 110 femtomolar (6.7×10⁷ virus particles per milliliter). This is the most sensitive method available to detect Ad5 binding. Using 4.8 mM NaCl gave maximum values of the Ad5 adsorption rate constant, k_{f}=1.3×10⁷(±4.7×10⁵) M⁻¹s⁻¹. Additionally, it has been found that increasing ionic strength decreased the magnitude of adsorption rate estimates. Diffusive mass transport to the surface limited Ad5 binding only at ionic strengths ≦9.6 mM. At 14.4 mM, the rate of Ad5 adsorption became comparable to the rate of diffusive mass transport.

Disclosed is a method to accurately and directly characterize interactions between biological materials using SPR technology. This is achieved by first assembling an intact resin or membrane on an SPR surface, then directly detecting the interaction between the biomolecule and the resin or membrane with SPR technology. From this data, estimates of mass-action ion-exchange adsorption and desorption rates accounting for steric and characteristic charge effects may be obtained. Previous methods using SPR technology only allowed measurement of minor aspects of the global interaction phenomenon, such as a single viral coat protein with a mammalian cell receptor. Based on these model studies, and many assumptions about the binding environment, researchers applied various mathematical models to determine estimated or theoretical binding rates. The improvement described herein allows detection and measurement of the binding of an entire viral particle, or other similar molecule, in a more natural environment, that of a three-dimensional membrane structure mimicking the membrane of mammalian cell walls. Furthermore, the invention enables detection and measurement of binding of viral particles, and other biomolecules, to commercially available isolation and purification materials commonly used in the bioscience research community, to allow more efficient preparation of medicaments for clinical use.

Commonly used isolation and purification techniques within the field of bioscience include column chromatography and the “batch method” of binding analytes to resins or other supports. Batch or column techniques only measure equilibrium binding constants which must be deconvoluted mathematically from effects of dispersion, diffusion or porosity. Furthermore, when attempting to purify or isolate an analyte for the first time, much precious material may be lost attempting to find the most effective and efficient isolation or purification method. In contrast, this new method allows adsorption and desorption kinetic rates to be measured directly under conditions very similar to flow operation of such large-scale techniques. The methods disclosed herein allow quantitative measurement of dynamic binding interaction between nanomolar to picomolar levels of precious analytes and their designated ligands, biological or synthetic, using SPR, whereas other commonly used techniques and methods, such as confocal microscopy, only qualitatively measures static binding outcomes at a scale of 100+nanometers. Furthermore, the method disclosed herein allows detection of actual binding of the biomolecule on real membrane or resins, something that in the past could only be inferred from qualitative data using several assumptions about the interactions that also required separate and often difficult if not impossible validation.

Interrogation of adsorption on three-dimensional surfaces by an exponentially-decaying evanescent wave that propagates perpendicularly from a plane that forms a boundary for the adsorptive surfaces requires a corresponding data reduction method to accurately estimate adsorption rate constants. The method disclosed herein permits accurate estimation of rate constants for uniform adsorption of a homogeneous analyte solution on homogeneous adsorptive sites distributed heterogeneously in space, relative to the planar boundary. Solutions are obtained for locally porous media and solid spheres. The method is extensible to other media and heterogeneous adsorptive sites.

The method of the invention as disclosed herein provides many advantages over currently existing methods used to detect and measure binding between analytes and designated ligands. Existing methods estimate adsorption rates for analytes or adsorptive sites with homogeneous or heterogeneous affinities. In current methods, geometric distribution of adsorptive sites relative to the planar origin of the evanescent wave is presumed to be uniform and one-dimensional. In contrast, the present method disclosed herein accounts for geometric heterogeneity in adsorptive-sites distribution. Current methods can only estimate adsorption rates accurately if adsorptive sites are distributed uniformly in the dimension perpendicular to the evanescent wave origin. In contrast, the method disclosed herein accurately measures uniform adsorption rates from analyte adsorption in spatially heterogeneous solid-liquid composite media adjacent to the planar origin of the evanescent wave. Current methods are derived primarily for proteins, whereas the present method disclosed herein is much more flexible and may be used for analytes of any size.

Mass transport limits binding kinetics when intrinsic reaction rates are fast relative to mass transport to the active surface (lateral mass transport), i.e., when kₐR_{T}≧kₘ. Increasing lateral mass transport can increase measurable values of sorption rate and surface site concentration that can be analyzed by dynamic surface adsorption measurement techniques like SPR or TIRF. Disclosed herein is a method to increase lateral mass transport by radial hydrodynamic diffusion (radial dispersion) by, for example, incorporating porous media such as a fixed fibrous bed or a concentrated packed bed of spheres in the SPR or TIRF flow cell.

The method disclosed herein provides many advantages over currently available techniques. Dispersion-enhanced lateral mass transport of adenovirus-sized particles occurs 4 to 10 times faster than diffusive mass transport for packed bed diameters between 10 and 50 microns for flow rates typical for biomolecular adsorption rate measurements by SPR. Dispersion-enhanced mass transport gives a boundary layer thickness δ=ξ(D/D_{∞}*)^{1/3} [28] while the steady-state concentration boundary layer thickness for free flow between two flat plates, δ corresponds to an order-of-magnitude average of ¾(DL/γ)^{1/3} [40]. For flowrates and diffusivities typical of biomolecular adsorption rate measurements by SPR, the ratio of these reduces to a simple function of system geometry independent of operating conditions:

$$\frac{{δ}_{Lok}}{{δ}_{Koch}} \approx {( \frac{Lh}{{a}^{2} ɛ} )}^{1 / 3}$$

For 10, 20 and 50 micron particles the ratio of free-flow to dispersion-enhanced boundary layers equals approximately 12, 7 and 4, respectively. Reduced boundary layer thickness occurs in conjuction with enhanced lateral mass transport.

This allows measuring non-specific electrostatic and hydrophobic adsorption on resin and membrane surfaces adjacent to the active plasmon resonant surface in which kₐ and R_{T} may be significantly larger than for specific biomolecular interaction, e.g. antibody-antigen interactions.

### DISCRIPTION OF THE FIGURES

FIG. 1. Structure and physical properties of Ad5. The Ad5 capsid is an 80-nm icosahedron composed of 12 different proteins. Fiber proteins form twelve 32-nm-long protrusions from 12 penton base vertices that mediate electrostatic interactions with cellular receptors. Hexon, penton and fiber proteins have isoelectric points ≦6.

FIG. 2. Physics of SPR molecular interaction measurement and analysis. (Adapted from [7]). The intensity of light reflected at a specific angle from a conducting gold film adjacent to the flow channel is decreased by resonant interaction with delocalized, coherently oscillating surface plasmons (I to II). The minimum angle shifts as the sample refractive index close to the glass surface changes when analyte binds to DEAE. The shift in angle due to adsorption over time is monitored as a resonance signal.

FIG. 3. Ad5 elution in a NaCl gradient from Resource™ Q column, as detected at A₂₅₄ by ultraviolet (UV) absorption spectroscopy. Resource Q is an anion exchange resin with terminal quarternary ammonium groups. Adenovirus primary capsid proteins hexon, penton and fiber have isoelectric points less than about 6.0 which gives the virus a net negative charge at physiological pH.

FIG. 4. Derivatization of the gold surface of the SPR Sensor. Sulfhydryl groups on 11-MUDA form covalent bonds with the gold surface. Adjoining van der Waals interactions result in a self-assembled monolayer. Terminal carboxylic acid groups form amide bonds with primary amines on diethylethylenediamine (DEEDA) after reaction with N-hydroxysuccinimide.

FIG. 5. Structures of chemical compounds involved in derivatization of gold surface. A schematic of the gold surface derivatization as shown in FIG. 4.

FIG. 6. Derivatization of gold surface plasmon resonance sensor monitored by refractive index adjacent to a derivatized gold film via SPR vs. time (in seconds). Steps in the process are noted in text boxes.

FIG. 7. Binding-elution profiles cytochrome c adsorption onto DEAE. Bold lines at each injection level are the result of simultaneously fitting all experimental data to obtain parameter values.

FIG. 8. SPR sensorgrams showing 0.52 pM Ad5 interaction with DEAE-SAM at 4.8 mM NaCl. Duplicate experiments are shown (solid lines). Data were fit to a two-compartment mass-transport limited model (bold line).

FIG. 9. SPR sensorgrams showing 1.0 pM Ad5 interaction with DEAE-SAM at 9.6 mM NaCl. The surface was regenerated to baseline values of plasmon resonant response units (RU) using 2.0 M NaCl. Duplicate experiments are shown (solid lines). Data were fit to a two-compartment mass-transport limited model (bold line).

FIG. 10. SPR sensorgrams showing 1.6 pM Ad5 interaction with DEAE-SAM at 14.4 mM NaCl. The surface was equilibrated with 10 mM HEPES pH 7.5, 14.4 mM NaCl. At time zero, 1.6 pM Ad5 in 10 mM HEPES pH 7.5 at 14.4 mM NaCl was introduced for 5 minutes. After 5 minutes, the surface was again equilibrated with 10 mM HEPES pH 7.5, 14.4 mM NaCl for an additional 5 minutes. Subsequently, the surface was regenerated to baseline values of RU using 2.0 M NaCl. Duplicate experiments are shown (hollow squares; hollow diamonds). A control injection containing no Ad5 gave a baseline SPR response (filled squares).

FIG. 11. A plot showing the Ad5 deposition rate on DEAE-SAM at increasing ionic strengths compared to an a priori estimate of deposition limited only by diffusive mass transport (the line is not a fit to the data).

FIG. 12A. The ratio of lateral mass transfer induced by incorporating solids into the sensor relative to mass transfer in a conventional open-channel SPR flow cell increases with Reynolds number (Re), analyte size and packed-sphere diameter. Data for spheres of diameter 2.5-μm, 5-μm, and 10-μm incorporated into the flow cell.

FIG. 12B. The ratio of lateral mass transfer induced by incorporating solids into the sensor relative to mass transfer in a conventional open-channel SPR flow cell increases with Reynolds number (Re), analyte size and packed-sphere diameter. Data for fibers with 2.5-μm, 5-μm, and 10-μm screening distances are shown. Filled symbols identify km ratios calculated for cytochrome c (radius=1.55 nm) at the three increasing diameters. Hollow symbols identify kₘ ratios calculated for adenovirus (radius=40 nm) at the three increasing diameters.

FIG. 13. Deposition of virus or protein on a solid sphere incorporated into the flow cell is illustrated (not to scale). A self-adsorbed monolayer (SAM) a few nanometers thick is formed on the surface to prevent sorption. Operating at a Reynolds number (Re) of 0.6 in a bed of 10-μm spheres yields a diffusive boundary layer thickness, δ, for Ad5 (radius=40 nm) of 250 nm. Electromagnetic field that generates the SPR signal decays exponentially with a length constant of about 240 nm.

FIG. 14. A scanning electron microscope image of a membrane formed by sintering polymer beads approximately 1 μm in diameter. The bar is 5 μm. The membrane surface appears on the left-hand-side and the interior on the right-hand-side.

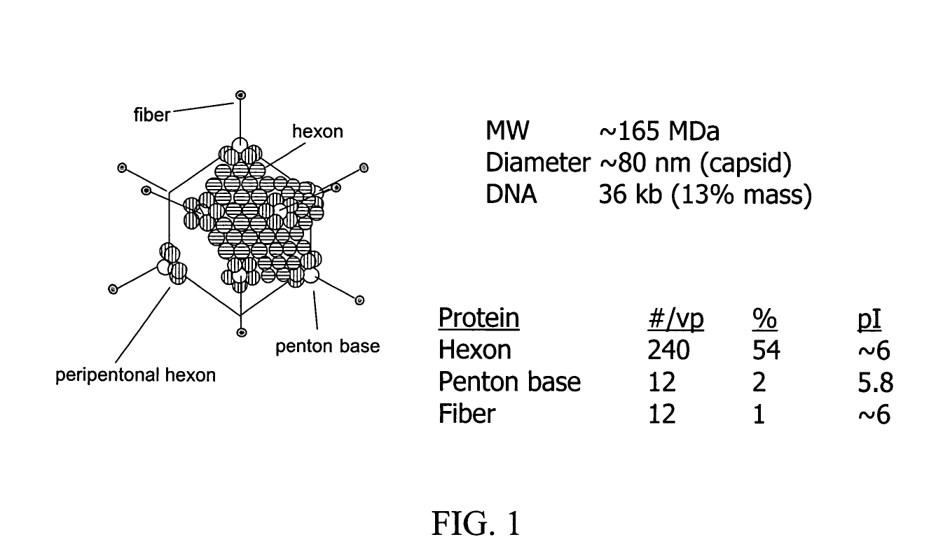

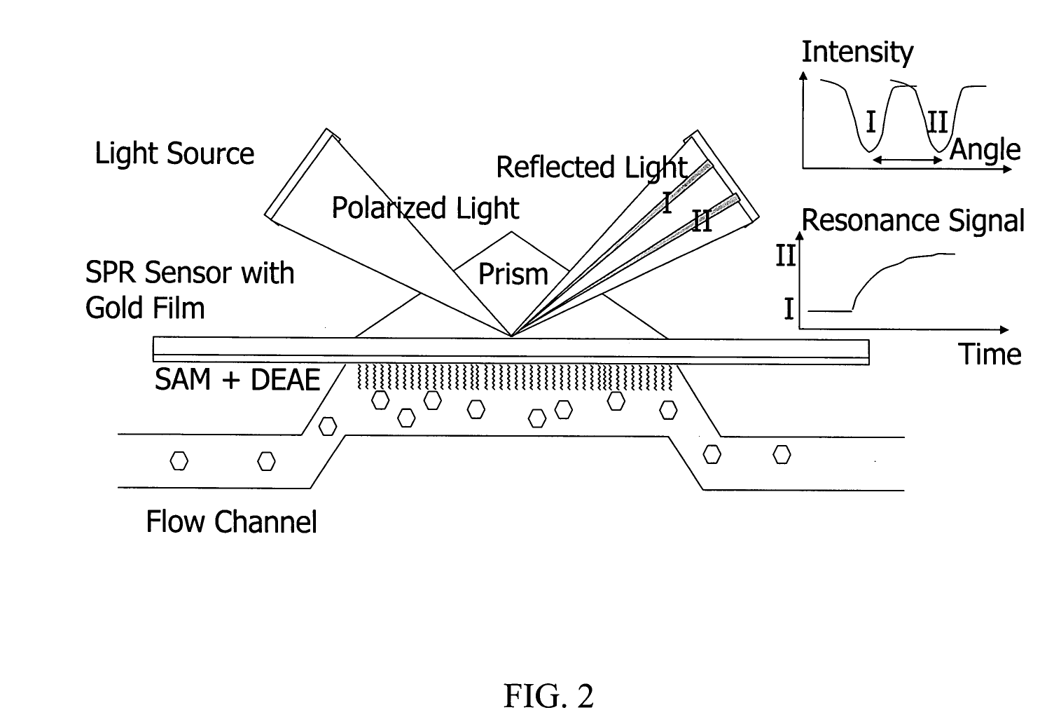

### DETAILED DESCRIPTION OF THE INVENTION

Herein, the effects of diffusive mass transport and ionic strength on nonspecific electrostatic interactions between DEAE modified surfaces and cytochrome c and Ad5 are reported. This pioneering invention is the first to enable measurement of sorption rate constants for nonspecific electrostatic interactions between protein or virus and a synthetic ion-exchange surface using SPR. Disclosed herein are binding kinetics of whole adenoviral particles, rather than isolated and purified capsid or fiber proteins.

Adsorption and desorption rate constants for nonspecific electrostatic interactions between cytochrome c and Ad5 and a synthetic ion-exchange surface, DEAE-substituted self assembled monolayer, have been measured using surface plasmon resonance. Binding kinetics of whole adenovirus were measured for the first time, rather than capsid or fiber proteins. These estimates were obtained at analyte concentrations near the limit of SPR detectability. Kinetic parameters were estimated for cytochrome c sorption on DEAE-substituted self-assembled monolayers (SAMs) down to 8.1 nM protein content. Estimates were obtained for Ad5-DEAE sorption in solutions as dilute as 520 femtomolar. This is believed to be the highest known sensitivity reported for detecting adenovirus binding interactions. Eliminating NaCl from the running buffer gave maximum values of the Ad5 adsorption rate constant, k_{f}=1.3×10⁷ (+4.7×10⁵) M⁻¹s⁻¹. Increasing ionic strength decreased the magnitude of adsorption rate estimates as well as the observed virus deposition rate on the surface.

To obtain these measurements, the gold SPR surface was derivatized with a MUDA-SAM and substituted with DEAE. N-hydroxysuccinimide (NHS) and N′-(ethylcarbonimidoyl)-N,N-dimethylpropane-1,3-diamine hydrochloride (EDAC) were used to catalyze amide bond formation between the terminal MUDA carboxyl group and the primary amine group of N,N-diethylethylenediamine (DEEDA). SPR monitored development of SAMs and inferred near stoichiometric yields during amide bond formation. Optimum regeneration of the DEAE-SAM surface to baseline SPR refractive index values between analyte injections was observed to take place by injecting 2.0 M NaCl and 40 mM sodium dodecyl sulfate (SDS). Ad5 was produced by infection of human embryonic kidney (HEK) cell cultures using aseptic technique. Ad5 recovery steps included freeze-thaw lysis of cells, centrifugal debris removal, Benzonase nucleic acid digestion, and purified by cesium chloride (CsCl) ultracentrifugation. Anion exchange high performance liquid chromatography (HPLC) with ultraviolet (UV) detection was used to quantify adenoviral titers using published methods as well as to recover purified Ad5 from residual CsCl.

The method disclosed herein permits accurate estimation of rate constants for uniform adsorption of a homogeneous analyte solution on homogeneous adsorptive sites distributed heterogeneously in space, relative to the planar boundary. Solutions are obtained for locally porous media and solid spheres. The method may be applied to other media and heterogeneous adsorptive sites. The method disclosed herein was performed as follows, SPR binding-elution responses were fit by a two-compartment mass-transport limited reaction model to get parameter estimates for cytochrome c and Ad5 electrostatic interaction with DEAE-SAMs. Surface heterogeneity terms accounted for variation in salt content between sample injection and running buffer in Ad5 sorption experiments. A binding energy estimated for cytochrome c from the measured dissociation constant was consistent with reported literature values. Forward rate constant values obtained for Ad5 were about 10-fold higher than values reported for interaction between Ad5 fiber knob protein and its cellular receptor (CAR). Reverse rate constants for Ad5 disassociation from DEAE-SAM were indistinguishable at the NaCl contents examined in the study.

SPR sensorgrams were fitted to a two-compartment mass transport limited reaction model using CLAMP software from the Center for Biomolecular Interaction Analysis at University of Utah (Salt Lake City, Utah, USA) to obtain rate constants. This model consisted of two coupled ordinary differential equations. In the model, analyte concentration in an outer compartment, C_{T}, is constant and equal to the injection concentration. Analyte concentration, C, in an inner compartment of height hᵢ is averaged over the length of the flow cell. Its value changes with time as analyte diffuses inward from the outer compartment and binds to immobilized DEAE to form complex B [11]:

$$\frac{ⅆ B}{ⅆ t} = {k}_{f} C ( {R}_{T} - B ) - {k}_{r} B \\ {h}_{i} \frac{ⅆ C}{ⅆ t} = {k}_{f} C ( {R}_{T} - B ) + {k}_{r} B + {k}_{m} ( {C}_{T} - C ) \qquad ( 1 a \& 1 b )$$

where RT represents the initial concentration of surface active sites. Other parameters are defined in the following text. This model is based on simple bimolecular interaction between analyte (A) and surface active site (RA) given by:

$$A + {R}_{A} {⟷}^{{k}_{f / {k}_{r}}} B \qquad ( 2 )$$

with forward (k_{f}) and reverse (kᵣ) rate constants. This model was augmented to account for diffusion-limited transport of analyte (virus or cytochrome c) to the gold sensor surface by:

$${A}_{T} {⟷}^{{k}_{m / {k}_{m}}} A \qquad ( 3 )$$

which has forward and reverse mass transfer coefficients (kₘ). These coefficients are equal and are related to flow cell length (L), width (b) and height (h), operational flow rate (O), and molecular diffusivity (D) by:

$${k}_{m} = 1.47 \cdot \sqrt[3]{\frac{{D}^{2} Q}{{h}^{2} bL}} \qquad ( 4 )$$

Values for L, b and h in the SPR flow cell of 5.0, 0.67, and 0.85 mm, respectively, were used to estimate the mass transfer coefficient [12].

Parameter values obtained from CLAMP for the experiments in this study were insensitive to the magnitude of the inner compartment height, hᵢ [11]. For convenience, hᵢ was set to the characteristic decay length of the evanescent plasmon resonant field, l_{d}=2.33×10⁻⁶ dm [13]. Physical properties of Ad5 capsid (R˜40 nm; MW=1.65×10⁶ Da) were applied to estimate maximum surface coverage, Bₘₐₓ, of 54.4 ng Ad5 per mm² corresponding to an initial surface active site concentration, R_{T}=3.3×10⁻¹² moles Ad5 per dm².

The SPR response factors for protein and adenovirus, respectively, were determined from the increase in refractive index, η, expected from adsorbed analyte (A) of radius R scaled by the maximum surface coverage, Bₘₐₓ, and a factor arising from the exponential decay of the evanescent field [13]:

$${R}_{f} = \frac{{10}^{6}}{{B}_{max}} ( {η}_{A} - {η}_{{H}_{2} O} ) [ 1 - exp ( - 4 \frac{R}{{l}_{d}} ) ] \qquad ( 5 )$$

In Eqn 5 the conversion factor of 10⁶ plasmon resonant response units (RU) per refractive index unit (RIU) has been applied [12,14]. Using a refractive index (RI) of η_{A}=1.57 for pure protein [13], Eqn 5 gives R_{f} values of 1.4×10¹⁰ RU per gram cytochrome c per cm² and 2.1×10¹⁰ RU per gram adenovirus per cm². The former is consistent with a surface coverage of ˜1 pg/mm² of protein on a two-dimensional surface that corresponds to a unit change in refractive index (RIU) of 10⁻⁶ [12,13,14].

CLAMP software was used in the following manner. SPR data in units of refractive index change vs. time were multiplied by a factor of 106 RU per RIU before being input into CLAMP. Fixed constants R_{T} and kₘ were divided by hᵢ as indicated in Eqn. 1a to give units of moles per dm³ and s⁻¹, respectively, before being used. R_{f} values were multiplied by molecular weight and hᵢ before being used to get 8.1×10¹⁰ RU/M (for virus) which gave consistent units. CLAMP was then run to fit Eqns. 1a and 1b to the data using only the two remaining parameters: rate constants k_{f}, and kᵣ with units of M⁻¹s⁻¹, and s⁻¹, respectively. The concentration of active sites, R_{T}, was used in addition to the rate constants to fit cytochrome c data.

The equilibrium constant for the bimolecular interaction of analyte and surface site for either interaction could be estimated by the dissociation constant, K_{d}=kᵣ/k_{f}. Using the dissociation constant in the van't Hoff expression gives the adsorption free energy:
ΔG_{ads}=RT ln K_{d}=ΔH−TΔS (7)
by applying the ideal gas constant, R, at absolute temperature, T. The lower the K_{d} values, the stronger the avidity of the analyte surface binding and the more negative the adsorption free energy.

The SPR response data appear consistent with a model of protein or virus-DEAE electrostatic interaction that is first-order in analyte concentration. Measured binding rates divided by Ad5 concentration remained constant as Ad5 concentration increased. Elution plateaus increased in proportion to Ad5 and cytochrome c concentrations, respectively as salt content in the sample matrix was kept constant. Binding slopes and elution profiles were elevated relative to analyte-free salt injection controls up to 14.4 mM NaCl. At 48 mM NaCl, adsorption of 0.11 pM Ad5 was undetectable.

Underlying data must conform to three implicit model criteria to obtain accurate estimates of intrinsic diffusive mass-transport and sorption rate constants by fitting two-compartment or effective rate models to SPR sensorgrams. First, the rate of surface reaction, k_{f}R_{T}, must be less than ten times the rate of lateral mass transport (normal to the sensor surface) [11] as the fitted model will be insensitive to sorption rate estimates 10-fold or more higher than the mass-transport rate. Second, aggregate concentrations of free and bound analyte normal to the sensor surface within the cuboid sensor region must be uniform. Otherwise, provided k_{f}R_{T}≦10*kₘ, effective rate coefficients that account for boundary layer height relative to mean free path are required [36]. Third, SPR responses must be accurately related to effective volume fractions and refractive indices of analyte, sorptive surface and solvent, respectively, distributed normal to the surface within the cuboid sensor region [13].

Mass transport coefficients were estimated using characteristic flow cell parameters, operating conditions and analyte diffusivity. For cytochrome c, adsorption and diffusion rates were comparable at ionic strengths lower than 4.8 mM. Comparing mass transport coefficients with adsorption rate constants revealed that diffusion limited Ad5 binding only at NaCl values ±9.6 mM. At higher ionic strengths, adsorption and diffusion rates for Ad5 were comparable. Comparing nonspecific Ad5 electrostatic adsorption rate constants measured in this study with literature value for receptor-mediated Ad5 interactions suggests Ad5 binding to cell surface receptors can be limited by adsorption rates rather than by diffusion at physiological values of ionic strength. The results suggest binding of Ad5 to ion-exchange media can be limited by adsorption rate rather than by diffusive mass transport at moderate ionic strengths.

Lateral mass transport in conventional SPR occurs only by molecular diffusion perpendicular to both laminar flow and to the surface under an adjacent boundary layer of thickness δ. Decreasing the height (h) of a SPR flow cell or increasing velocity increases diffusive mass transport proportional to ⅙, as km in Equation 4 increases in proportion to y Laminar-flow boundary layer thickness, [symbol] =O[¾(DL/γ)^{1/3}], decreases in proportion to the ⅔ power of decreases in flow cell height, since [symbol] =6F/h²b. For example, the 2×10⁻³ cm height of the new Biacore 3000 (Biacore AB, Uppsula, Sweden) flow cell gives a value of [symbol] ˜54% as large as the Biacore 2000 sensor (h=5×10-3 cm and L=0.24 cm). This corresponds to a 1.8-fold increase in lateral mass transfer and an equivalent 1.8-fold increase in the range of measurable k_{f} or R_{T} values.

The novel method outlined here minimizes boundary layer resistance and maximizes lateral mass transport relative to conventional laminar flow cells (described by Equation 4) by stimulating transverse hydrodynamic diffusion [34]. Transverse hydrodynamic diffusion (also called transverse or lateral dispersion) accompanies velocity fluctuations caused by disturbances due to submerged solids that produce fluid eddies large relative to the mean free diffusive path. Eddy mixing chaotically extinguishes uniform parallel streamlines of laminar flow and produces random motion both perpendicular and parallel to the direction of macroscopic flow [35]. Transverse hydrodynamic diffusion transports mass to sensor surfaces faster than molecular diffusion by orders of magnitude and substantially reduces the thickness of the diffusive boundary layer adjacent to the surface.

Transverse hydrodynamic dispersion can be stimulated by inserting solid surfaces whose cross-sections are normal to flow in the sensor cell. Interactions between hydrodynamic dispersion and the sensing region, boundary layer, sphere diameter and analyte size are now examined. Solid surfaces spheres or fibers differ in the degree to which they enhance transverse hydrodynamic diffusion. We quantify the degree of enhancement using a mass transfer coefficient for porous media derived from scaling analysis [28]:
kₘ=(3^{3/2}[d₁′D_{∞}*D²]^{1/3})/(4πξ+3^{3/2}h[d₁′D²]^{1/3}D_{∞}*^{-2/3}) (8)
In equation 8, D_{∞}* is the transverse hydrodynamic diffusion coefficient, ξ is the Brinkman screening distance, and d₁′=O(1)3200/9π² is a dimensionless constant where O(1) is a constant on the order of unity. The Brinkman screening distance is obtained implicitly for a fibrous bed from
a²/ξ²=((10Φ)/(3[ln(ξ/a)]+ln(2)−0.577216))+(O(Φ/(ln³(1/Φ))) (9)
where [symbol] is solids volume fraction, and α is the radius of particulate fibers or particles. For a packed bed of spheres, ξ=α.

Expressions to determine D_{∞}* far from the wall (z>>ξ) are available for four types of porous media: packed spheres, fibers, highly permeable monodisperse solids and highly-permeable bidisperse solids. For packed spheres [29]:
D_{∞}*=((2a(F/(εhb))/(Pe_{f}))+(D/τ) (10)
where Pe_{f}=40-29exp(−7/Re) for spheres, τ=1.2 is the tortuosity measured in the transverse direction and ε is the bed porosity. The Reynolds number is Re=2aUₒρ/ [symbol] with Uₒ being the superficial velocity. For dilute isotropic fiber beds in the limit of high Peclet number, D_{∞}*=9π²Ua/(3200ξΦ). Transverse hydrodynamic diffusion for highly permeable monodisperse and bidisperse media can also be calculated using available expressions [30].

Higher lateral mass transport is accompanied by a reduction in boundary layer thickness, δ. When δ<<mean free diffusive path of a ligand in the boundary layer before a binding event occurs, the concentrations of free and bound ligand in the boundary layer normal to the surface are essentially uniform (second model criterion). Uniform concentrations permit ligand binding onto 3-D surfaces in the boundary layer within the SPR active sensing region to be modeled as binding on a flat surface. So enhancing lateral transport of diffusion-limiting macromolecules to reduce boundary layer thickness is also necessary to apply the two-compartment model to accurately fit SPR sensorgrams from sorption on 3-D surfaces.

In particular, binding to 3-D surfaces in the sensing region within the diffusive boundary layer can be modeled as binding on a flat surface provided boundary layer thickness is less than the distance traveled by a ligand to a binding site. The distance traveled by a ligand is its mean free path [36]:
x=(((1−Φ)D)/k_{f}R_{3D}))^{1/2}
where Φ and R_{3D} are the effective solid-phase fraction and effective volumetric concentration of receptors, respectively, in the boundary layer. The ratio of boundary layer thickness to mean free path is then
δ/x=((k_{f}Rδ)/((1-Φ)D))^{1/2} (12)
where R═R_{3D}δ is the effective surface concentration of free receptors. Wofsy and Goldstien (2002) [36] showed rate constants determined from 3-D binding data differ from intrinsic rate constants by <10% if the ratio of boundary layer thickness to mean free path is <0.5 or if (1−Φ)D/(kₘδ)>2.

Enhanced lateral hydrodynamic diffusion allows intrinsic sorption rates to measured directly on planar or 3-D surfaces in the sensor region by fitting standard models, provided SPR response factors accurately represent exponentially decaying evanescent signal interacting with 3-D structures and distal adsorption. Relationships are developed elsewhere for SPR response to effective volume fractions and refractive indices of analyte, sorptive surface and solvent, respectively, distributed normal to the surface within the sensor region for planar and 3-D surfaces [37,34]. Operating conditions that enhance lateral mass transport and maintain uniform ligand concentration in the boundary layer are now defined to enable SPR sensorgram data obtained to be fit by the convenient two compartment model.

Incorporated solid surfaces could induce weak variations in decay length due to local changes in effective refractive index and cause nonidealities in SPR response such as increased dip-angle width and secondary minima. But forming homogeneous, close-packed solid structures on sensor surfaces that enhance minimum percent reflectivity have been shown to minimize dip-angle width and eliminate secondary minima [38]. Homogeneous deposition of monodisperse polystyrene spheres on a sensor produced minor surface roughness and unimodal SPR profiles [39]. Variations in decay length can also be minimized by homogeneous deposition, small surface coverages or adlayer thicknesses, or matched refractive index values of solid and solvent [37].

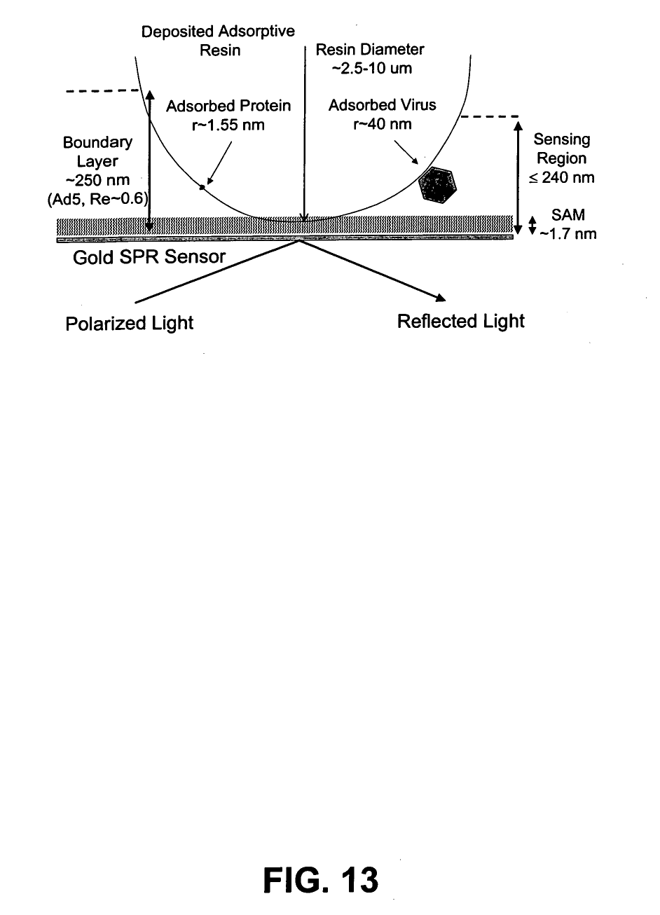

Jung et al. (1998) [13] established a quantitative formalism consistent with Maxwell's equations that relates SPR response to adsorbate refractive index, film thickness and coverage on planar SPR surfaces. Nonuniform 1-D coverage by analytes much smaller than the exponentially decaying evanescent field length was considered, but SPR responses to adsorption on 3-D surfaces or to discontinuous distribution of analyte perpendicular to a planar SPR surface were not discussed. Ramsden et al. (1994) [41] used a similar formalism to evaluate optical waveguide sensor response due to adsorbed cells of complex shapes. The present disclosure extends the quantitative formalism to characterize SPR response from adsorption on 3-D structures and from discontinuous distribution of analyte perpendicular to the planar SPR surface a graphical example of which is provided in FIG. 13.

SPR response, R_{f}, occurs as a shift in wavelength or angle of the minimum in reflected light intensity that corresponds to a time-dependent change in effective refractive index of the medium adjacent to the metal sensor surface, η_{eff}(t) relative to an initial (solvent) refractive index, ηₛ:
R_{f}(t)=m₁(η_{eff}(t)−ηₛ)+m₂(η_{eff}(t)−ηₛ)² (13)
where m₁ is a sensitivity factor varying from ˜3100 to 8800 nm/RIU for a planar SPR spectrometer. The quadratic term in Equation 13 is negligible for small changes in refractive index.

The effective refractive index, η_{eff}(t), in Equation 13 consists of refractive indices of adsorbing analyte, η_{A}, and solvent, ηₛ in the sensing region weighted by changing surface coverage, θ(t), and term arising from the SPR signal source. SPR signal is generated by an evanescent electromagnetic field that decays away exponentially into the medium in the z-direction perpendicular to the sensor surface and exhibits a characteristic decay length, l_{d}. The measured refractive index perpendicular to the sensor surface is therefore also weighted by the light intensity, or square of the field strength: exp(−2z/l_{d}). The effective index of refraction is then determined by integrating the intensity-weighted refractive index over the depth of the interrogated field,
η_{eff}(t)=(2/l_{d})∫₀^{∞}η(t,z)exp(−2z/l_{d})dz (14)
Jung et al. (1998) [13] analyzed only continuous Heaviside functions of η(t,z), such as monolayer adsorption of analyte, A, with radius r on a planar surface at a particular time. Planar monolayer adsorption yields a bilayer refractive index: η(z)=η_{A} for 0≦z≦2r and η(z)=ηₛ for z>2r. This expression for the bilayer refractive index may be substituted into Equation 14 and the result substituted into Equation 13 to obtain the SPR response:
R_{f}=m₁(η_{A}−η_{S})[1−exp(−4r/l_{d})] (15)
The sensitivity factor, m₁, in Equation 15 may be evaluated by scaling a conversion factor of 10⁶ SPR response units (RU) per refractive index unit (RIU) by the maximum surface coverage, Qₘₐₓ, to give 10⁶/Qₘₐₓ [14].

The formalism relating SPR response to parameters of planar SPR surfaces is now extended to analyte adsorption on 3-D surfaces within the sensing region and discontinuous distribution of analyte normal to the sensor surface. In any plane parallel to the sensor surface at distance z within the cuboid sensing region, contributions to spatially varying refractive index, η(t,z), from analyte, η_{A}(z), solid particles, ηₚ(z) or solvent, ηₛ(z), are proportional to the area fraction of analyte, θ(t,z), solid Φ(t,z) or solvent (1−θ(t,z)−Φ(t,z)) in that plane, respectively, viz:
η(t,z)=η_{A}(z)θ(t,z)+ηₚ(z)Φ(t,z)+(1−θ(t,z)−Φ(t,z))ηₛ(z) (16)
Discretizing η(t,z) into planar coordinates x and y is inordinate unless performing SPR imaging, since SPR response constitutes an average obtained from the cuboid sensing region. Contributions to η(t,z) from distributions along x and y coordinates are projected onto and the z-axis in the sensor and its mathematical description. Using the present approach, Equation 16 permits discontinuous functions of η(t,z) normal to the sensor surface to be analyzed. This will be illustrated in the cases that follow.

The time-varying expression in Equation 16 may be substituted into Equation 14 to obtain the measurable effective refractive index of the cuboid sensing region relative to an initial refractive index that consists of either solvent or solid, or both solvent and solid. The refractive index change is then inserted into Equation 15 to relate SPR responses to area fractions and refractive indices of analyte, sorptive solid and solvent, respectively, in 3-D analyte distribution and absorption. Two cases of immediate practical interest are examined: (1) analyte deposition onto a homogeneous anisotropic porous adsorbent adjacent to the sensor surface (Example XVII); and (2) analyte deposition onto close-packed solid spheres adjacent to the sensor surface (Example XVIII). Response factors from these cases are applicable to a variety of porous media that may be used to obtain SPR adsorption-rate measurements. Porous membranes formed by sintering granular polymer beads, for example, form layers of micron-scale particles arranged homogeneously at the membrane surface.

Development of these two cases neglects weak variations in decay length due to local changes in η_{eff}(t) and nonidealities in SPR response related to incorporating media such as increased dip-angle width and secondary minima. Dip-angle width is minimized and secondary minima eliminated by forming a homogeneous, close-packed solid structure that also enhances minimum percent reflectivity [38]. Homogeneous deposition of monodisperse polystyrene spheres on a sensor has produced minor surface roughness and unimodal SPR profiles [39] Variations in decay length can also be miminized by homogeneous deposition, small surface coverages or adlayer thicknesses, or matched refractive index values of solid and solvent [13].

In one embodiment of the present invention, the method disclosed herein is used to detect and measure binding interactions of an analyte with a three-dimensional surface.

In another embodiment of the present invention, the method disclosed herein is used to detect and measure binding interactions of an analyte with a self-assembled, close-packed, ordered monolayer.

In another aspect of the invention, large biomolecules on the order of ≦10⁶ Da, such as viral particles, may be detected adsorbing and desorbing to such intact surfaces, such as a membrane that mimics a mammalian cellular outer membrane surface.

It is another aspect of the invention that the intact three-dimensional surface be composed of various materials including, but not limited to, chemicals that mimic the outer surface of mammalian cellular membrane surface, self-assembled, close-packed, ordered monolayer surfaces made of various resins used in large-scale isolation and purification techniques, such as, but not limited to, ion exchange resins based on DEAE structures, and other porous or non-porous polymer-based resins.

In yet a further embodiment of the present invention, to better detect binding of analytes using SPR, or other optical technology, whose mass transport limits binding kinetics when intrinsic reaction rates are fast relative to mass transport to the active surface (lateral mass transport), improvements in said lateral transport are disclosed herein through the use of radial hydrodynamic diffusion (radial dispersion) by, for example, but not limited to, incorporating porous resin media such as a fixed fibrous bed or concentrated packed bed of spheres to create said continuous three-dimensional surface within the SPR or TIRF flow cell.

An additional embodiment of the present invention constitutes use of disclosed novel mathematical models used to interrogate adsorption on three-dimensional surfaces using an exponentially-decaying evanescent wave that propagates perpendicularly from a plane that forms a boundary for the adsorptive surfaces, such as, but not limited to, SPR technology. The models provide accurate estimation for rate constants for uniform adsorption of homogeneous analyte solutions on homogeneous adsorptive sites distributed heterogeneously in space (three-dimensionally) relative to the planar boundary. The method examples may make use of locally porous media and solid spheres, for example, but the method is extendable to other media and heterogeneous adsorptive sites.

In a more preferred embodiment, the method disclosed herein allows direct detection and measurement of the efficiency of binding of viral particles to various isolation and purification media or resin using SPR technology.

In a further preferred embodiment, the method disclosed herein allows direct detection and measurement of the efficiency of binding of large biomolecules to intact membranes or continuous surfaces of resin or media and the like in three-dimensional space using a mathematical model to estimate the said binding efficiency.

In a further preferred embodiment, the new method disclosed herein allows direct detection and measurement of the efficiency of binding of large biomolecules to intact membranes or continuous surfaces of resin or media and the like in three-dimensional space using a mathematical model to estimate the said binding efficiency, wherein only very small quantities, in the nanomolar or femtomolar range, of the analyte being studied is required to obtain the information regarding binding of the analyte to the chosen surface using SPR technology.

To further illustrate the invention without limiting it, the following illustrative examples are provided.

### EXAMPLES

### Example I

Cell Culture and Propagation

Human Embryonic Kidney cells (P/N 293 HEK; ATCC, Rockville, Md.) at a concentration of 1×10⁶ cells/mL were inoculated in 5 mL of DMEM purchased from Sigma (St. Louis, Mo., US). The medium was supplemented with 0.1 g/l alanine, 0.110 g/l sodium pyruvate, 1 g/l glucose, 0.584 g/l L—glutamine, 37 g/l sodium bicarbonate—all from Sigma (St. Louis, Mo., US)—and 10 ml/l antibiotic from Gibco (Auckland, NZ), pH 7.8. Cells were incubated in T-flasks from Corning (Corning, N.Y., US) at 37° C. and 5% CO₂ for 48-96 hours. Flasks with cells at 90% confluence were split 1:5 into additional T-flasks to propagate the cell line. Near-confluent cells were resuspended by striking the flask 6-10 times, subdivided between 4 additional flasks and supplemented with fresh culture media to nurture new cell growth. Alternatively, cells at 90% confluence were also infected with Ad5.

### Example II

Adenovirus Infection and Propagation

To infect the cells, a 1:100 dilution of Ad5 from ATCC (Rockville, Md., USA) was added to confluent T-flasks without disrupting adherent cells. T-flasks were incubated 1 hr at 37° C. and 5% CO₂. After one hour, the cells are supplemented with additional culture media. After 48-72 hours the cytopathic effect was observed and virus was harvested. T-flasks were agitated to resuspend all cells. Suspension was centrifuged 5 min at 3,000×g. Supernatant was removed and combined with 10% glycerol before storage at −70° C. for future infection. Recovered cell pellets were resuspended in equal volumes of Tris buffer, pH 7.8, +1 mM CsCl, both from Sigma (St. Louis, Mo., US). The resuspension was frozen at −70° C. then thawed (repeated three times) to release Ad5. After 3× freeze-thaw, resuspensions were centrifuged to remove cell debris, then treated with Benzonase for 30 minutes to digest nucleic acid. Digested supernatants were ultracentrifuged atop low- and high-density CsCl bands to purify viral capsids.

### Example III

Adenovirus Chromatography

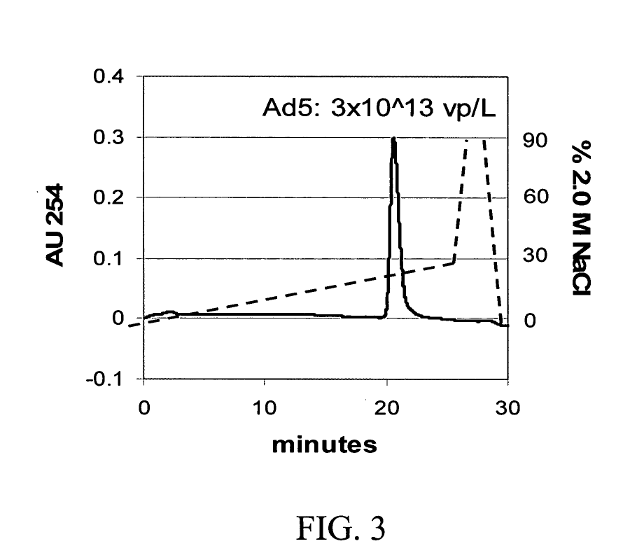

Ad5 was be purified and analyzed by HPLC with UV detection using Resource™ Q anion exchange resin from Amersham Biosciences (Piscataway N.J., US). The chromatogram is shown in FIG. 3. A quaternary ammonium anion exchange media, Resource Q, distinguished Ad5 from byproducts with a NaCl gradient (dotted line) from 40 to 600 mM at 1 mL/min, using the fact that hexon, penton and fiber proteins have IEP 6. A static capacity of ˜5×10¹¹ virus per mL, a detection limit >1×10⁸ particles per milliliter, and a linear Ad5 virus particle/HPLC area ratio of 20,085 have been reported for this method. [5]

### Example IV

Surface Plasmon Resonance Derivatization

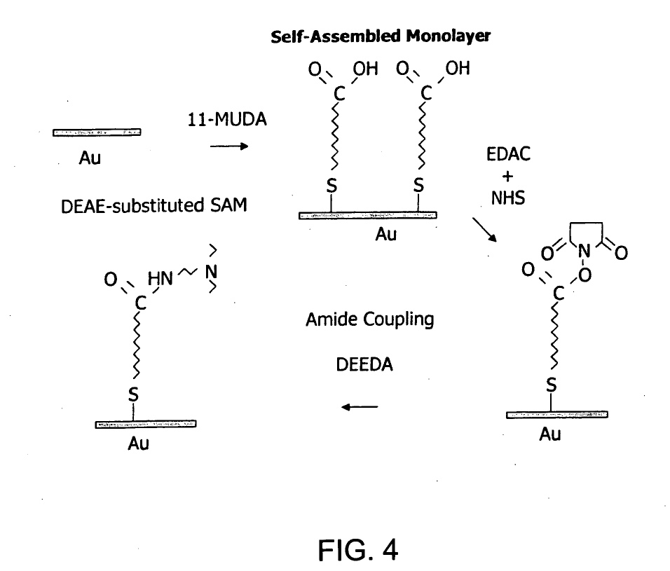

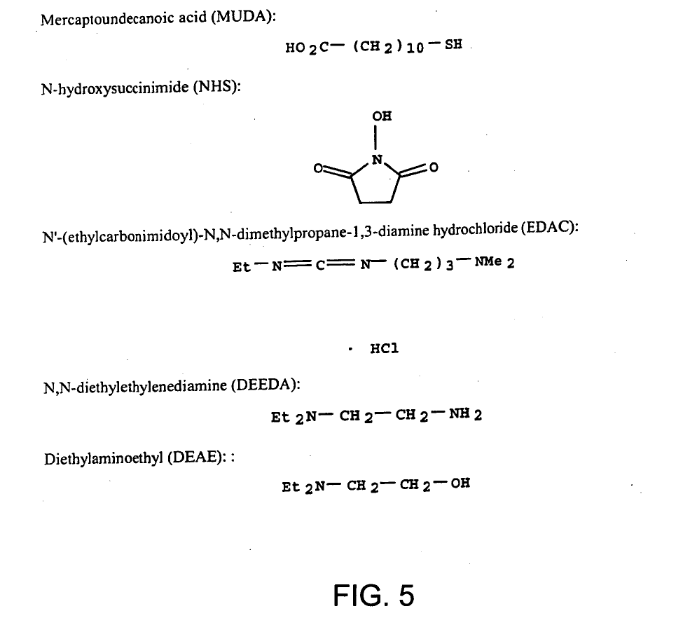

Derivatization with mercaptoundecanoic acid linked to terminal diethylethylenediamine was performed as follows. A schematic of this process is shown in FIG. 4. All chemicals were obtained from Sigma (St. Louis, Mo., US) unless otherwise noted. Structures of chemical compounds are shown in FIG. 5. The gold sensor surface was calibrated in deionized, distilled water. The surface was cleaned with 0.1 M NaOH and 1% Triton™, then equilibrated in 10 mM MES, pH 5.0 (3.0 ml/h). It was equilibrated in degassed ethanol, then exposed to 2.0 mM 11-mercaptoundecanoic acid (MUDA) in degassed ethanol until refractive index stabilized (6-24 hrs; 0.1 ml/h). The self-assembled monolayer of MUDA was rinsed with ethanol (0.6 ml/h), then 10 mM MES pH 5.0 (3.0 ml/h) until refractive index stabilized. Freshly prepared solutions of 0.4 M EDAC and 1.0 M NHS in 10 mM MES were mixed 1:1 and injected for 20 minutes (3.0 ml/h) to prepare the terminal carboxyl group for amide bond formation. 1.0 M diethylenediamine (DEEDA) in 0.01 M MES pH 5 was added for 0.75-2 hrs (3.0 ml/h). The surface was rinsed with 10 mM MES pH 5, then rinsed with 0.01 M MES pH 6.7 (6.0 ml/h).

### Example V

Surface Plasmon Resonance Measurements

An SPR instrument including an integrated flow cell, gold-coated sensors, and software from Nomadics, Inc (Stillwater, Okla., US) was attached to a syringe pump from Cole Parmer, Inc (Vernon Hill, Ill., US), via a manual PEEK injection valve from Upchurch, Inc (Oak Harbor, Wash., US) using PEEK tubing and fittings. All binding assays were carried out at 25° C. Refractive index baselines were established using 10 mM MES pH 5.0 or 10-50 mM HEPES pH 7.5 running buffers. NaCl from 0.0048 M to 0.048 M was added to the running buffer, depending on the experiment. Flow rate was maintained at 6 ml/hr to optimize the sorption profiles. Ad5 or cytochrome c was diluted into running buffer and injected onto the SPR system via the injection valve from either the sample loop or a syringe using a second syringe pump for 5 minutes. The surface was then washed with running buffer for an equivalent period of 5 minutes to study dissociation of the analyte from the surface. The surface was regenerated between injections to baseline RU values using successive injections of 2 M NaCl alternated with 0.04 M SDS. Attempted regeneration with NaOH, HCl and/or Triton™ reduced the apparent mass derivatized on the surface, as measured by change in baseline refractive index, coincident with a subsequent reduction in apparent adsorptive capacity, also measured by change in refractive index.

### Example VI

Derivatization Chemistry of Gold Surface Plasmon Resonance Sensor

A schematic of the derivatization of the gold surface of the SPR sensor is shown in FIG. 4. Structures of each compound involved are shown in FIG. 5. The sulfhydryl group of 11-mercaptoundecanoic acid (MUDA) interacts with adsorbed AuCl₂⁻ anions on the gold sensor to self-assemble a close-packed, ordered monolayer (SAM) that is 1.7+/−0.2 nm thick, due to van der Waals and hydrophobic forces among the carbon chains.[15] The terminal carboxyl group of the MUDA-SAM is derivatizable via formation of amide bonds, catalyzed by N-hydroxysuccinimide (NHS) and N′-(ethylcarbonimidoyl)-N,N-dimethylpropane-1,3-diamine hydrochloride (EDAC). Amide bond formation between the terminal MUDA carboxyl group and the primary amine of N,N-diethylethylenediamine was selected to model anion-exchange of Ad-5 on DEAE-substituted surfaces.

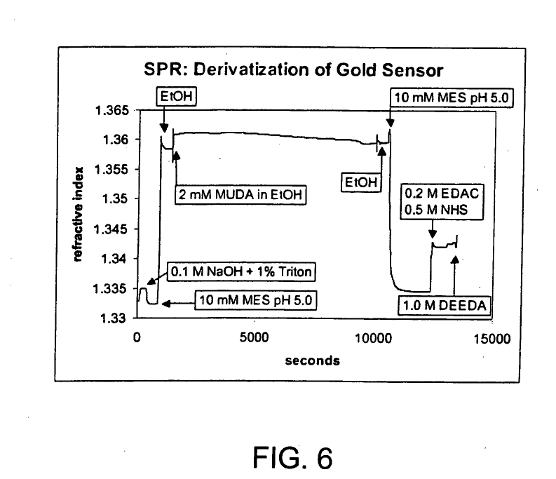

FIG. 6 shows refractive index changes monitored during the derivatization, up to the addition of DEEDA. Refractive index increases with addition of cleansing solution, ethanol, MUDA solution EDAC/NHS and DEEDA. It decreases upon flushing with 2-morpholinoethanesulfonic acid, monohydrate (MES) buffered aqueous solutions. Refractive index values after derivatization subtracted from baseline values before derivatization were used to estimate masses of MUDA SAM, EDAC/NHS and DEEDA adsorbed to the gold. The surface was derivatized with 2180 RU of MUDA (218.36 Da) and 1290 RU of DEEDA (116.21 Da). This corresponds to a ˜1:1 stoichiometric mass ratio of the two components.

The gold surface was cleaned with 0.1 M NaOH and 1% Triton™, then equilibrated in 10 mM MES, pH 5.0 (3.0 ml/h). It was equilibrated in degassed ethanol, then exposed to 2.0 mM 11-mercaptoundecanoic acid (MUDA). The self-assembled monolayer of MUDA was rinsed with ethanol, then 10 mM MES. Freshly prepared solutions of 0.4 M EDAC and 1.0 M NHS in 10 mM MES were mixed 1:1 and injected to prepare the terminal carboxyl group for amide bond formation. 1.0 M diethylenediamine (DEEDA) in 0.01 M MES pH 5 was added, marking the end of monitoring.

### Example VII

Sorption of Cytochrome c to DEAE-Substituted SAM

To characterize the DEAE-substituted SAM surface we monitored sorption profiles of cytochrome c. Cytochrome c (12,400 Da; 2R=3.1 nm) is a globular, well-characterized biological redox protein. At pH 7.5, cytochrome c (pI 10.2) has a positive charge of about +8, calculated from its amino acid sequence and the heme.[16] Acidic patches on its surface due to aspartate (D) and glutamate (E) residues [17] are primarily responsible for its electrostatic interaction with the anionic DEAE-substituted MUDA layer[18] DEAE-substituted materials are fully positively charged only below pH 5. The diethylaminoethyl group has a pKa of 9-9.5. However, substantial titration of DEAE-substituted materials is observed around pH 6-8 due to electrostatic repulsion of protons from closely adjacent substituents that affect local pKa values[19]

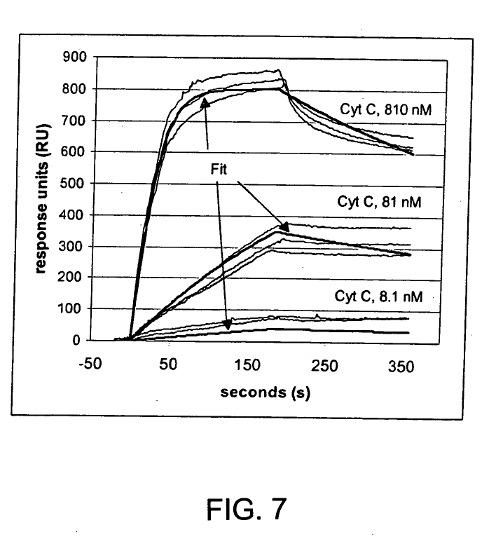

FIG. 7 shows the SPR sensorgrams for interaction between cytochrome c and DEAE-substituted SAM. In each experiment, the surface was equilibrated in 10 mM N-2-hydroxyethylpiperazine-N′-2-ethanesulfonic acid (HEPES) buffer at pH 7.5. Cytochrome c was injected at 300 μl/min at concentrations of 0.01 mg/ml (8.1×10⁻⁴ mM), 0.001 mg/ml (8.1×10⁻⁵ mM) and 0.0001 mg/ml (8.1×10⁻⁶ mM), respectively. All solutions were prepared in 10 mM HEPES pH 7.5. The refractive index rise during association phase and decay during dissociation phase was consistent with protein adsorption to, and desorption from, the surface. The surface was regenerated to its pre-injection refractive index (RI) value between injections using 2 M NaCl and 40 mM SDS. That SDS improved surface regeneration suggests hydrophobic interactions between adjacent cytochrome c molecules or between cytochrome c and the self assembled monolayer may also occur. This would not be surprising, considering the large hydrophobic patches on the surface of cytochrome c and the hydrophobic nature of the C11 backbone of 11-MUDA.

Parameter estimates for cytochrome c interaction with DEAE-SAM were obtained by simultaneously fitting experimental data from all injection levels to a two-compartment mass-transport limited model. Fitted curves are shown as bold lines in FIG. 7. Values of the resulting parameter estimates are summarized in Table I. The binding of cytochrome c to DEEDA derivatized on the gold surface can be characterized by on rate constant, k_{f}=6.9±0.053×10⁴ M⁻¹s⁻¹ and off rate constant kᵣ=1.8±0.033×10⁻³ s⁻¹. Errors shown are standard deviations from the different experiments in FIG. 7. These values lead to an average dissociation rate constant, K_{d}=2.6×10⁻⁸. Forward and reverse mass transfer coefficients were equivalent and calculated to be kₘ=5.5×10⁻⁴ cm s⁻¹=6.8×10⁷ RU(Ms)⁻¹ using Equation. 4.

TABLE I
Parameter Estimates for
Cytochrome C Interactions with DEAE-SAM

| | | Cytochrome C |
|---|---|---|
| Buffer NaCl | mM | 0 |
| Sample NaCl | mM | 0 |
| Cytochrome C | nM | 8.1, 81, 810 |
| Q | μl/min | 300 |
| kₘ × 10⁻⁷ | RU (Ms)⁻¹ | 6.8 |
| k_{f} × 10⁻⁴ (± std. dev.) | M⁻¹s⁻¹ | 6.9 ± 5.2 × 10² |
| kᵣ × 10³ (± std. dev.) | s⁻¹ | 1.8 ± 3.3 × 10⁻⁵ |
| K_{d} × 10⁸ | M | 2.6 |
| B × 10⁻² (± std. dev.) | RU | 8.3 ± 2.0 |

The physical properties of cytochrome c (R=1.55 nm; MW=12,400 Da) can be applied to estimate a maximum surface coverage of 2.7 ng cytochrome c per mm² (2.2×10⁻¹³ mol cytochrome c per mm²). This coverage corresponds to a maximum of 2.2×10⁻⁹ adsorptive sites per dm 2 or 2728 RU, using the conversion factor of 1 RU per 10⁻¹⁰ g/cm protein. The fitted value for adsorptive sites of 828 RU is 30% of the estimated maximum site value. Similarly, the observed increase in refractive index plateaus at 8.3×10⁻⁴ units after 5 minutes exposure to 0.01 mg/ml (8.1×10⁻⁴ mM)cytochrome c. This plateau is 31% of the value of 2.7×10³ RIU expected for maximum surface coverage[13] Because equilibrium was not reached in individual binding assays during the time of injection it was not possible to compute a complete binding isotherm. This precluded Scatchard analysis.

By employing surface heterogeneity in the fit of cytochrome c-DEAE interaction data to the two-compartment mass-transport limited model, fitted response curves could be obtained to more closely reflect the curvature during desorption (data not shown). Assuming surface heterogeneity provided the three additional fitting parameters shown in Eqn. 6, k′_{f}, k′ᵣ, and B′, and reduced the residual sum squared error from 32.4 to 28.6. Whether this modest improvement in fit justifies the additional complexity of a heterogeneous model remains under investigation.

### Example VIII

Comparison with Literature Values of Cytochrome C Sorptive Interactions

Because cytochrome c is cationic at physiological pH values, its purification by DEAE chromatography occurs infrequently[20,21] Values of kinetic rate or equilibrium constants for the cytochrome c-DEAE interaction could not be found in the literature, but estimates for free energy of cytochrome c adsorption onto hydrophilic glass have been reported: −13.37 kcal/mol [22] and −11.8 kcal/mol [23]. Using the SPR-measured dissociation rate constant, K_{d}=2.6×10⁻⁸, and Eqn. 7, we estimate a free energy of adsorption for cytochrome c interaction with DEAE to be ΔG_{ad}=−10.3 kcal/mol. This value appears consistent with the previously reported values for electrostatic adsorption of cytochrome c.

### Example IX

Cytochrome C-DEAE Interaction: Mass Transport Effects

Mass transport limits electrostatic adsorption when the reaction rate is fast relative to diffusive mass transport. This occurs when k_{f}R_{T}>>kₘ, given that these parameters have consistent units. We chose this set of units to evalute the parameters: k_{f}[=] M⁻¹s⁻¹, RT [=] M cm and kₘ [=] cm s⁻¹. Substituting k_{f}=6.9×10⁴ M⁻¹s⁻¹ and R_{T}=6.7×10⁻¹⁰ M-dm obtained from SPR measurements gives a value of k_{f}R_{T}=4.6×10⁻⁴ cm s⁻¹. The mass transport coefficient was estimated using Eqn. 4 to be kₘ=5.5×10⁻⁴ cm s⁻¹. From these estimates, it appears that molecular diffusion to the surface and electrostatic adsorption at the surface in the plasmon resonant system occur at similar rates. Neither rate can be neglected in the analysis.

### Example X

Sorption of Adenovirus Type 5 to DEAE-Substituted SAM: SPR Sensorgrams—Electrostatic Interaction Between Ad5 and DEAE

Ionic strength was observed to affect parameter estimates for electrostatic interaction between Ad5 and DEAE. Ionic strengths from 4.8 to 48 mM NaCl and Ad5 concentrations from 0.52 to 1.6 pM Ad5 were evaluated. SPR sensorgrams detected Ad5 interaction with DEAE-modified SAM at ionic strengths between 4.8 and 14.4 mM NaCl in the injected sample, but not at 48 mM NaCl.

TABLE II
Ad5 Interaction with DEAE-SAM

| | | Ad5 | Ad5 | Ad5 |
|---|---|---|---|---|
| Eluent NaCl | mM | 4.8 | 9.6 | 14.4 |
| Sample NaCl | mM | 4.8 | 9.6 | 14.4 |
| Ad5 | pM | 0.52 | 1.0 | 1.6 |
| Q | μl/min | 300 | 300 | 300 |
| kₘ × 10⁵ | cm s⁻¹ | 5.1 | 5.1 | 5.1 |
| Response × 10⁻¹⁰ | RU/M | 8.1 | 8.1 | 8.1 |
| k_{f} × 10⁻⁶ | M⁻¹s⁻¹ | 13 | 2.9 | 1.6 |
| (± std. dev.) | | (±0.47) | (±0.030) | (±0.011) |
| kᵣ | s⁻¹ | 0 | 0 | 0 |
| B | pM · dm | 3.3 | 3.3 | 3.3 |

### Example XI

Sorption of Adenovirus Type 5 to DEAE-Substituted SAM: SPR Sensorgrams—Improved Detectability of the Ad5-DEAE Interaction

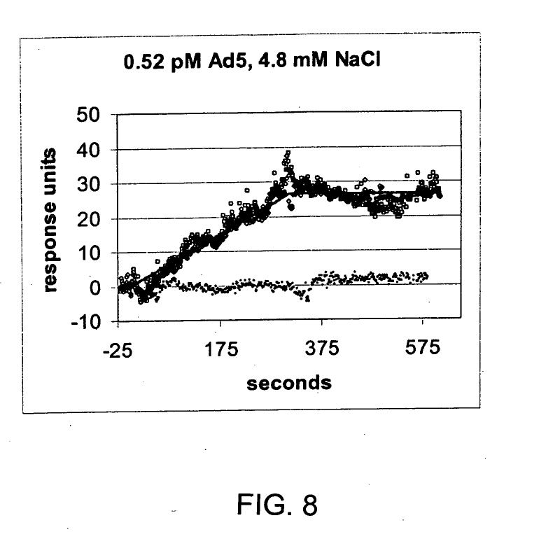

Adding salt to the running buffer and increasing the Ad5 concentration improved detectability of the Ad5-DEAE interaction. FIG. 8 shows duplicate binding-elution profiles of 0.52 pM Adenovirus Type 5 sorption onto DEEDA fit to a two-compartment mass-transport limited model. The surface was equilibrated in 0.0048 M NaCl and 0.01 M HEPES pH 7.5. A sample containing 0.52 pM Ad5 in approximately 0.0048 M NaCl, 0.01 M HEPES pH 7.5 was applied to the surface at 300 μl/min for 5 minutes. Upon introducing the sample there is a linear increase to 27 RU as Ad5 adsorbs to DEAE in the sensor cell. Introducing elution buffer stops binding of Ad5 to DEAE-derivatized surface.

### Example XII

Sorption of Adenovirus Type 5 to DEAE-Substituted SAM: Ad5-DEAE Interaction Data Fit to the Two-Compartment Model

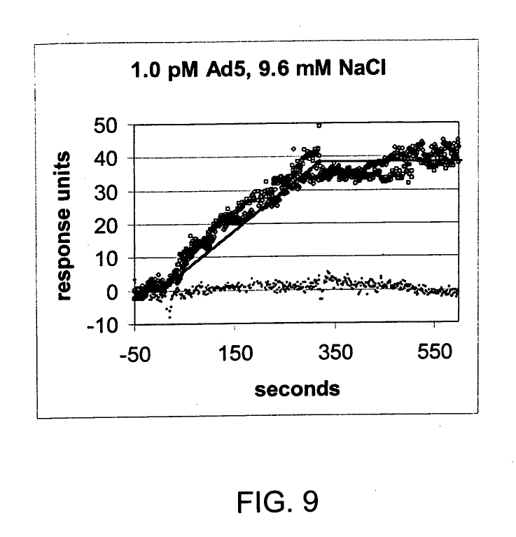

FIG. 9 shows duplicate binding-elution profiles of 1.0 pM Ad5 sorption onto DEAE fit to the two-compartment model. The surface was equilibrated in 0.0096 M NaCl and 0.01 M HEPES pH 7.5 and a sample containing 1.0 pM Ad5 in approximately 96 mM NaCl, 0.01 M HEPES pH 7.5 was applied to the surface at 300 μl/min for 5 minutes. Upon introducing the sample there is a linear increase to 38 RU due to Ad5 binding. The rate of RU increase per unit time obtained from fitting the respective data sets is only 1.5 times larger for 1.0 pM Ad5 than for 0.52 pM Ad5. It appears that increasing ionic strength has lowered the relative binding rate of Ad5.

### Example XIII

Sorption of Adenovirus Type 5 to DEAE-Substituted SAM: Ad5-DEAE Interaction Data Fit to the Two-Compartment Model with Increased NaCl

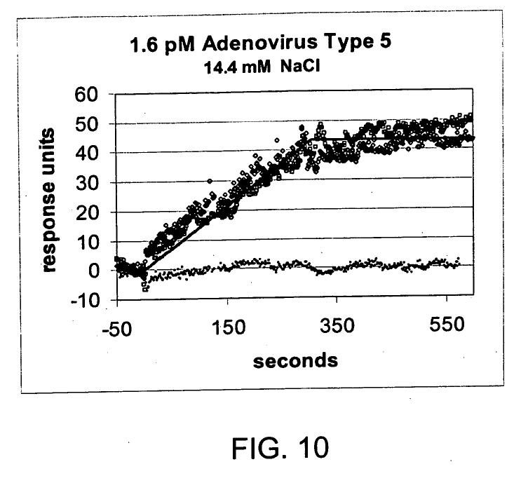

Further increases in Ad5 and NaCl concentration did not increase in SPR detectability. FIG. 10 shows duplicate binding-elution profiles of 1.6 pM Ad5 sorption onto DEAE fit to the two-compartment model. The surface was equilibrated in 0.0144 M NaCl and 0.01 M HEPES pH 7.5. A sample containing 1.6 pM Ad5 in approximately 14.4 mM NaCl, 0.01 M HEPES pH 7.5 was applied to the surface at 300 μl/min for 5 minutes. Upon introducing the sample there is a linear increase to 44 RU due to Ad5 binding. The rate of RU increase per unit time obtained from fitting the respective data sets is only 11% larger for 1.6 pM Ad5 than for 1.0 pM Ad5, suggesting ionic strength is decreasing the deposition rate.

At 48 mM NaCl, interactions between 0.11 pM Ad5 and the DEAE-SAM were not detectable by our SPR method (data not shown). At ionic strengths >30 mM, Debye lengths decrease to nanometer levels and surface heterogeneities become important. These conditions may preclude electrostatic Ad5 interaction with DEAE-SAM. Alternatively, binding of these dilute virus solutions may not be measurable due to large refractive index effects of high background salt content.

### Example XIV

Ad5-DEAE Interaction Parameter Estimates

Table II summarizes parameter estimates from Ad5 interaction with DEAE-SAM at increasing values of Ad5 and NaCl concentration. Trends consistent with electrostatic interaction between Ad5 and DEAE are observed. The estimated forward rate constants for Ad5-DEAE binding decrease as NaCl content in the eluent increases. Reverse rate constants for nonspecific electrostatic Ad5-DEAE interaction are undistinguishable at the ionic strengths examined.

### Example XV

Ad5-DEAE Interaction: Colloid and Mass Transport Effects

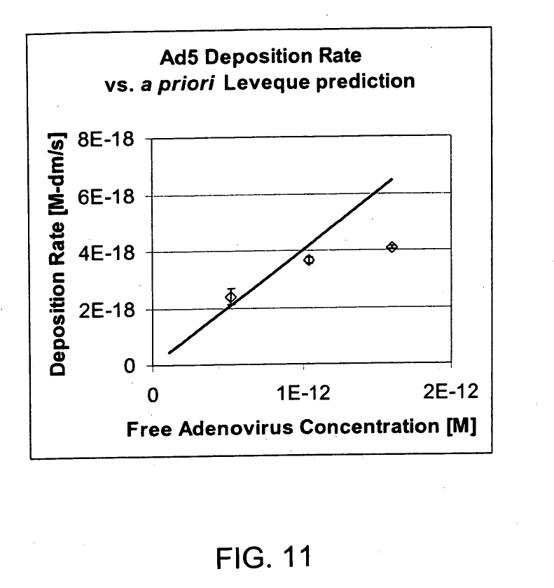

FIG. 11 shows deposition rate data at three different Ad5 and NaCl concentrations compared with an a priori prediction of deposition with an infinitely fast adsorption rate. At 14.4 mM, observed deposition rates were markedly lower than predicted rates, indicating that at this ionic strength, finite adsorption rate values limit binding to the surface and are no longer negligible relative to diffusive mass transport to the surface.

Mass transport limits binding when the electrostatic interaction rate is fast relative to diffusive mass transport, i.e., when k_{f}R_{T}>>kₘ. We select the following consistent set of units for the analysis: k_{f} [=] M⁻¹s⁻¹, R_{T} [=] M cm and kₘ [=] cm s⁻¹. Substituting the fitted k_{f} values and the estimated adenovirus R_{T}=3.3×10⁻¹² M dm gives k_{f}R_{T}=4.3×10 cm s⁻¹ for 0.52 pM Ad5, 9.6×10⁻⁵ cm s⁻¹ for 1.0 pM Ad5 and 5.3×10⁵ cm s⁻¹ for 1.6 pM Ad5. The Ad5 mass transport coefficient estimate is kₘ=5.1×10⁻⁵ cm s⁻¹.

Comparing these values reveals that at 4.8 mM NaCl adsorption occurs 8.2 times faster than mass transport, resulting in diffusive mass-transport limited binding. Increasing NaCl to 9.6 mM reduces the relative adsorption rate to 1.9 times the mass transport rate. At 14.4 mM, the adsorption rate is essentially equivalent to n diffusive mass transport. It is significant that only at ionic strengths 9.6 mM or lower does diffusive mass transport limit nonspecific interaction between Ad5 and DEAE-modified surfaces. This is somewhat surprising, considering the size and low diffusivity of Ad5. However, it is consistent with previous observations that fowl plague virus binding to suspended chick embryo cells occurred at one-third the rate predicted by Fick's Law for Brownian collisions between the species[24] These data confirm that diffusive and electrostatic interaction rates must both be considered even at modest ionic strengths when evaluating nonspecific virus binding.

The trend observed in this data suggests that as ionic strength increases to a value near physiologic salt content (150 mM), adsorption rates could in fact limit binding of Ad5 to DEAE surfaces. Interestingly, Garnier et al. concluded that adsorption controls Ad5 interaction with HEK cells after estimating a dissociation constant of K_{d}=5.3×10⁻¹² M from a model of virus diffusion through a finite boundary layer [25]. That model assumed reversible monovalent virus-cell receptor binding at the liquid-cell interface, pseudo steady-state flux, constant total cell surface receptor number and equilibrium between fluxes at the interface.

### Example XVI

Comparison with Literature Values of Adenovirus Type 5 Sorptive Interactions

Consider parameter estimates for nonspecific Ad5-DEAE kinetic rate constants relative to values reported in the literature for biospecific receptor-ligand interactions between Ad5 fiber knob and CAR receptor. Table III contains reported estimates for on and off rate constants and dissociation constants for interaction between Ad5 fiber knob and the N-terminal immunoglobulin domain D1 of the coxsackievirus and adenovirus receptor (CAR D1), the soluble extracellular domain of CAR (s-CAR) and its immunoglobulin domain (IgV) obtained using SPR. An estimate of K_{d}=65 nM was obtained from a Scatchard analysis of saturated binding of Ad3 dodecahedron containing 12 penton bases to a ubiquitin protein ligase measured by SPR [26]. The NaCl content for reported literature values is 150 mM. This is about ten to thirty times larger than the NaCl content in this study. One reason off rate constants were unmeasurable in this study was likely because of the low concentration of NaCl. Forward rate constants for biospecific Ad5 fiber knob protein interactions are about 10 to 50 times lower than nonspecific electrostatic interaction between Ad5 and DEAE-SAM. This comparison suggests that receptor-mediated Ad5 binding to cell surfaces can be adsorption-rate limited, rather than diffusion limited.

TABLE III
Ad5 fiber knob interactions with CAR receptor

| | | L.-Jacob^{[8]} | L.-Jacob^{[8]} | Kirby^{[9]} | Kirby^{[9]} |
|---|---|---|---|---|---|
| Buffer NaCl | mM | 150 | 150 | 150 | 150 |
| Sample NaCl | mM | 150 | 150 | 150 | 150 |
| System | | SPR | SPR | SPR | SPR |
| Injected | | CAR D1 | Ad2 knob | Ad5 knob | Ad5 knob |
| Immobilized | | Ad2 knob | CAR D1 | s-CAR | IgV |
| Ad knob | nM | | 2-50 | 10-5000 | 10-5000 |
| Q | μl/min | 10 | 20 | 5-40 | 5-40 |
| kₘ × 10⁻¹⁰ | Ms⁻¹ | Na | na | na | na |
| k_{f} × 10⁻⁶ | M⁻¹s⁻¹ | 0.25 | 0.24 | 0.18 | 0.062 |
| (±std. dev.) | | (±0.02 × 10⁵) | (±0.04 × 10⁵) | (±0.02 × 10⁵) | (±5.4 × ⁴) |
| kᵣ × 10³ | s⁻¹ | 6.6 | 5.5 | 2.8 | 1.4 |
| (±std. dev.) | | (0.01 × 10⁻³) | (±0.01 × 10⁻³) | (±0.07 × 10⁻³) | (±1.3 × ⁻³) |
| K_{d} | nM | 26.4 | 23 | 14.8 | 10.4 |
| B × 10⁻³ | RU | 1.2 | 0.2 | | |
| (±std. dev.) | | (±1.3 × ⁷) | (±6.4 × 10⁵) | | |

### Example XV

Calculating 10-Fold Increases in Ranges of Measurable Rate Constants and Surface Capacities

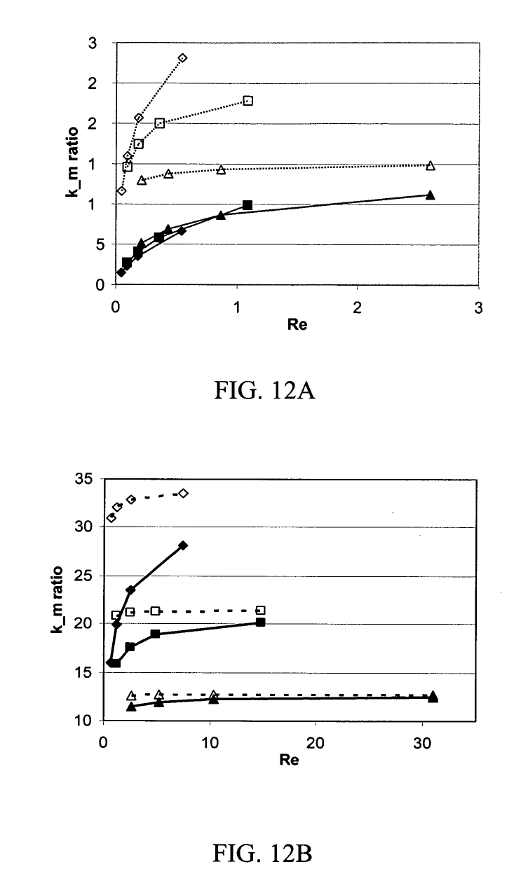

The ratio of lateral mass transfer coefficient in porous media (Equation 8) to the mass transfer coefficient in a standard open-channel flow cell (Equation 4) under comparable conditions (the kₘ ratio) identifies increases expected in measurable ranges of rate constants and surface capacities. Mass transfer coefficients in Equation 8 that change for different values of transverse hydrodynamic diffusion coefficient, D_{∞}*, and Brinkman screening distance, ξ, corresponding to packed spheres, fibers, and highly permeable monodisperse and bidisperse solids will shift the kₘ ratio for different types of porous media. FIG. 12A shows the kₘ ratio (el) for flow cells where lateral dispersion is enhanced by incorporating packed spheres. The kₘ ratio increases with Re and with analyte size (cytochrome c and Ad5 have radii of 1.55 and 40 nm, respectively). The kₘ ratio also increases as sphere diameter decreases from 10 to 2.5 μm. Incorporating 2.5-μm spheres and operating at Re≧2.5 increases lateral transport of cytochrome C≧10-fold relative to a conventional open channel. A similar increase is expected for Ad5 by incorporating 2.5- or 5-μm spheres and operating at Re≧0.6. The range of measurable rate constants or surface capacities in both cases would be ≧10-fold relative to measurements in an open channel.

Lateral dispersion could also be enhanced using isotropic fibers with Φ=0.375. FIG. 12B shows 10-fold enhancements at all Re≧0.6 for fiber diameters between 2.5 and 10 μm. Effects of Re, analyte size, and fiber diameter on km ratio are consistent with those for packed spheres. Similar calculations for permeable and highly-permeable monodisperse media show comparable enhancements. Table IV summarizes order-of-magnitude enhancements in lateral mass transport from four kinds of media for adenovirus adsorption at Re˜7 by incorporating solids with a characteristic dimension of 2.5 μm. Each of these solids at these conditions substantially increases the range of measurable intrinsic sorption rates or surface capacities relative to open-channel SPR.

### Example XV

Calculating Boundary Layer Thickness Reductions Due to Transverse Hydrodynamic Diffusion

Reductions in boundary layer thickness due to enhanced transverse hydrodynamic diffusion can be calculated using relations for δ in open-channel and solids-containing flow cells. Steady-state concentration boundary layer thickness for laminar flow between two flat plates, δₗₐₘ, corresponds to an order-of-magnitude average of ¾(DL/γ)^{1/3} [27]. Boundary layer thickness when lateral mass transport is enhanced by incorporating solids is δ_{Disp-Enhance}=ξ(D/D_{∞}*)^{1/3} [28]. These expressions give a ratio of laminar-flow to dispersion-enhanced boundary layer:
δₗₐₘ/δ_{Disp-Enhance}=(3/(4ξ))((LD_{∞}*)/γ)^{1/3} (13)
where γ=6F/h²b, ξ is found using Equation 9 for fibers (ξ=a for solid spheres) and D_{∞}* is found using Equation 9 for solid spheres, or corresponding expressions for fibrous, monodisperse or bidisperse media.

Substituting flow-cell values of F=3 cm³ s⁻¹, L=0.24 cm, b=0.02 cm and h=0.005 cm into Equation 13 shows that for adenovirus mass transfer through 2.5, 5- and 10-μm packed spheres, the ratio of laminar-flow to dispersion-enhanced boundary layer equals approximately 10, 7 and 5, respectively. For 2.5-μm spheres, Eq 12 shows this new method allows accurate sorption rate measurements for virus that have 10-fold lower diffusivities or 10-fold higher sorption rates than existing procedures. Alternatively, a sensor with 10-fold more receptor sites or a 10-fold lower fluid phase fraction could be used.

Applying expressions for ξ and D_{∞}* suited to isotropic fibers with these diameters shows corresponding boundary layer reductions of 23, 14 and 9. Similar reductions are obtained for highly permeable monodisperse and bidisperse beds. Table IV summarizes order-of-magnitude reductions in boundary layer thickness expected from four media types for adenovirus adsorption at Re˜7 using solids with a characteristic dimension of 2.5 μm.

TABLE IV
Increase in lateral mass transport and reduction in boundary layer
thickness by incorporating media into the SPR sensor.

| | Fold Increase in | | Correlations for |
|---|---|---|---|
| | Lateral Mass | Fold Reduction in | Transverse |
| | Transport | Boundary Layer | Hydrodynamic |
| | (kₘ ratio)¹ | Thickness² | Diffusion |
| Solid Spheres | 22 | 10 | Gunn, 1987 [29] |
| Fibers | 33 | 23 | Gunn, 1987 [29] |
| Monodisperse | 38 | 17 | Moutsoloplis and |
| | | | Koch, 1999 [30] |
| Bidisperse | 16 | 14 | Moutsoloplis and |
| | | | Koch, 1999 [30] |

¹Calculated from Eqs 4 & 8 for Ad5 adsorption at Re˜7 using a characteristic dimension of 2.5-μm.
²Calculated using Eq 13 for Ad5 adsorption at Re˜7 using a characteristic dimension of 2.5-μm.

### Example XVI

Calculating Operating Conditions and Geometries to Measure Sorption of Adenovirus and Cytochrome C

Adenovirus Type 5 (Ad5) is a non-enveloped, double-stranded DNA viral vector used in gene therapy to treat diseases such as cancer, diabetes, hemophilia, cystic fibrosis, heart disease and musculoskeletal disorders that have an underlying genetic basis [31]. Ad5 has a capsid radius of about 40 nanometers, a molecular mass of 165×10⁶ daltons and a molecular diffusivity of 4.5×10⁻⁸ cm²s⁻¹ [32]. Ad5 vectors for gene therapy are prepared by adsorption onto ion-exchange chromatographic resin, for which kinetic adsorption rates are desired. Cytochrome c is a globular, well-characterized biological redox protein with a molecular mass of 12,400 daltons, a diameter of 3.1 nanometers and a molecular diffusivity of 1.6×10⁻⁶ cm²s⁻¹ [33].

FIGS. 12A and 12B show that lateral mass transfer enhancement for Ad5 and cytochrome C is determined by choice of operating conditions and system geometry: Re, sphere/fiber diameter and analyte diameter. Inserting 2.5-μm spheres into a flow cell and operating at Re>0.6 is expected to increase lateral mass transport >18-fold for Ad5 and >6-fold for cytochrome c. Intrinsic rate constants for sorption of Ad5 can be extracted using the two-compartment model provided uniform-concentration criteria, δ/x<0.5 or (1−Φ)D/(kₘδ)>2, are met. Substituting an adsorption rate of k_{f}R_{T}=2.5×10⁴ cm s⁻¹ [34] and other physical parameters into Eq 12 shows operating at Re>0.006 or Re<0.005 meet the respective criteria for Ad5. In this calculation the effective fluid-phase fraction in the boundary layer was estimated as 1−Φ=0.68 at Re˜0.006 using geometric considerations, including an area fraction of 0.9069 for hexagonal closed packed (HCP) 2.5-μm spheres at the plane that intersects their radii. Operating at Re>0.006 or Re<0.003 satisfied the two respective criteria for cytochrome c adsorption using 2.5-μm spheres. Without enhancing transverse hydrodynamic diffusion, neither of the above criteria was met for 2.5-μm resin at Re values less than 3.8 with virus. Broader ranges of acceptable particle size and Re were possible for cytochrome c than for virus due to its higher molecular diffusivity.

Enhanced transverse dispersion produced by 2.5-μm spheres at Re>0.6 allows intrinsic adsorption rates of k_{f}≦10*kₘ/R_{T} to be measured using the two-compartment model. This corresponds to an upper limit of k_{f}≦5.1×10⁹ M⁻¹s⁻¹ which is obtained using km=9.1×10-3 cm/s, calculated from Equation 8, 9 and 10, and R_{T}=1.8×10⁻¹² moles Ad5 per dm², calculated from physical properties of Ad5. This limit is >18 times higher than what is currently measurable using conventional open-channel SPR sensor with planar surfaces. This analysis could be applied to make analogous calculations fibrous, monodisperse, or bidisperse media.

### Example XVII

Analyte Deposition onto a Homogeneous Anisotorpic Porous Absorbent Adjacent to the Sensor Surface.

Analyte deposition onto a homogeneous anisotropic porous media with solids fraction Φ, total capacity Rₜₒₜ=R*δ and uniform boundary layer fluid-phase concentration. To obtain uniform boundary layer fluid-phase concentration, diffusion time to the surface must be less than diffusion time to adsorptive sites. In this case, any plane a vertical distance, z, from the sensor surface has analyte area θ(z,t)*Lb and solids area Φ(z)*Lb. The factor θ(z,t) corresponds to the probability that analyte adsorbs at vertical distance z and time t. The fraction of fixed, homogeneous solid media in this case is time-invariant. For homogeneous anisotropic porous media, solid area on every plane may be projected onto a sub-plane of area Φ*Lb, Φ being independent of z. Similarly, analyte adsorbing on every plane at any moment from inception to equilibrium may be projected completely onto a sub-plane of area θ(t)*Lb. The effective refractive index is then the sum of each of these projected areas weighted by their Analyte deposition onto a homogeneous anisotropic porous media with solids fraction Φ, total capacity Rₜₒₜ=R*δ and uniform boundary layer fluid-phase concentration. To obtain uniform boundary layer fluid-phase concentration, diffusion time to the surface must be less than diffusion time to adsorptive sites. Methods to achieve uniform boundary layer concentration are considered elsewhere (Roper 2004). In this case, any plane a vertical distance, z, from the sensor surface has analyte area θ(z,t)*Lb and solids area Φ(z)*Lb. The factor θ(z,t) corresponds to the probability that analyte respective refractive indices and by the exponentially decaying light intensity. Substitution into Equation 16 and rearranging gives:
η_{eff}−ηₛ=(η_{A}−ηₛ)θ(t)+(ηₚ−ηₛ)Φ (17)
after integrating the exponential weight factor from z=0 at the sensor surface to z=∞ to get l_{d}/2, since Φ and θ(t) are independent of z.

The SPR signal is then proportional to the appropriate refractive-index difference: either (η_{eff}−ηₛ) after depositing the homogeneous anisotropic porous media onto a clean gold sensor surface with θ(t)=0; or (η_{eff}−ηₛ−(ηₚ−ηₛ)Φ) after exposing analyte to the porous media adjacent to the gold sensor surface. The latter result yields
R_{f}(t)=m₁(η_{A}−η_{H2O})θ(t) (18)
The SPR signal in Equation 18 is proportional to the uniform partition coefficient 0(t) and is not decreased by [1−exp(−2d/l_{d})] as was the case in Equation 15 for adsorption directly onto a planar, possibly derivatized, gold sensor surface. This expression is applicable to ligand interacting with receptors derivatized onto polymer matrices when analyte diffusion time to surface is less than its diffusion time to an adsorptive site on the polymer. Analyte area fraction θ(z,t) may be related geometrically to solids area fraction Φ(z) as will be shown in Example XVIII.

### Example XVII

Analyte Deposition Onto Close-Packed Solid Spheres Adjacent to the Sensor Surface

Analyte of radius r deposited from a uniform boundary layer fluid-phase concentration onto solid spheres of radius R that are hexagonally close-packed (HCP) onto the surface. Area void at the radius of the HCP spheres is ε=0.0931. A uniform boundary-layer fluid-phase concentration implies diffusion time to the surface is less than diffusion time to sphere adsorptive sites. Define dimensionless distance χ=z/R normal to the sensor surface, dimensionless analyte-to-adsorbent ratio ζ=2r/R and dimensionless weighting factor ν=2R/1_{d}. Geometrical considerations show that for small ζ and small ζν/4, any plane a distance χ from the sensor surface has analyte area fraction θ(χ,t)=(1−ε)ζ(2+ζ)f(t) and solids area fraction Φ(χ)=(1−ε)(2χ−χ²). Time-dependent analyte deposition, f(t) varies from 0 to approximately the hard-sphere RSA jamming limit of 0.546. Substituting these terms into Equations 16 and 14, respectively, then integrating and combining terms gives:

$$\begin{matrix}{η}_{eff} ( t ) - {η}_{s} = \frac{2}{υ} ( 1 - ɛ ) ( 1 - \frac{1}{υ} ) ( {η}_{p} - {η}_{s} ) + \\ ( 1 - ɛ ) ζ ( 2 + ζ ) f ( t ) ( {η}_{A} + {η}_{s} )\end{matrix} \qquad ( 19 )$$

The SPR signal is then proportional to the appropriate refractive-index difference given by Equation 19. This difference is (η_{eff}−ηₛ) after adding solid particulate to a clean gold sensor surface with f(t)=0 which renders 2^{nd} term on the right-hand-side of Equation 19 zero. Or it is [η_{eff}−ηₛ−(2/ν)(1−ε)(ηₚ−ηₛ)] after exposing analyte to fixed solid spheres adjacent to the gold sensor surface. The dimensionless analyte/adsorbent ratio 4 varies from about 0.08 for sorption of 40-nm virus on 1-micron-scale sintered membranes to ˜0.0004 for small protein adsorption on 10-micron beads.

### Example XIX

Measuring Adsorption of Adenovirus and Cytochrome C on Planar Surface, Membrane, and Resin

Adenovirus Type 5 (Ad5) is a non-enveloped, double-stranded DNA viral vector used in gene therapy to treat diseases such as cancer, diabetes, hemophilia, cystic fibrosis, heart disease and musculoskeletal disorders that have an underlying genetic basis [31]. Ad5 has a capsid radius of about 40 nanometers, a molecular mass of 165×10⁶ daltons and a molecular diffusivity of 4.5×10⁻⁸ cm²s⁻¹ [32]. Ad5 vectors for gene therapy are prepared by adsorption onto ion-exchange chromatographic resin, for which kinetic adsorption rates are desired. Cytochrome c is a globular, well-characterized biological redox protein with a molecular mass of 12,400 daltons, a diameter of 3.1 nanometers and a molecular diffusivity of 1.6×10⁻⁶ cm²s⁻¹ [16].

First, monolayer adsorption of analyte on a planar sensor surface is considered. Substituting pure protein refractive index of η_{A}=1.57, a decay length of l_{d}=240 nm [98] into Equation 15 and applying physical properties of Ad5 yields an R_{f} value of 2.2×10¹⁰ RU per gram adenovirus per cm. An R_{f} value of 1.4×10¹⁰ RU per gram cytochrome c per cm² calculated in the same manner is consistent with a surface coverage of ˜1 pg/mm² of protein on a two-dimensional surface that corresponds to a unit change in refractive index (RIU) of 10⁻⁶ [14]. FIG. 10 shows SPR sensorgrams of 1.6 pM Ad5 (14.4 mM NaCl) deposition onto a planar sensor derivatized with DEAE-SAM obtained using this response factor. Also shown is an Ad5-free control injection. Refractive index increases linearly to 43.6 RU during the 5-minute adsorption period and remains near this value during the subsequent desorption period.

Second, analyte deposition onto homogeneous anisotropic porous media from a uniform-concentration boundary layer without adsorption onto the planar surface of the sensor will be considered. The porous media could be a polymer layer or highly permeable monodisperse or bidisperse media. Comparing Equations 18 and 15 shows SPR response to this deposition is θ(t)/(1−exp(4r/l_{d})) times the SPR response to monolayer surface adsorption. Let porous media of Φ=0.2 extend to the decay length of the evanescent wave and assume θ=0.05, which corresponds to a modest capacity of about 50 mg/ml surface-associated analyte in the media, and set operating conditions for uniform concentration. Equation 18 yields R_{f} values of 2.7×10¹⁰ RU per gram cytochrome c per cm² and 2.2×10⁹ RU per gram adenovirus per cm², respectively. These values indicate SPR sensitivity increases 2-fold for deposition of cytochrome c, but decreases by about 90% for deposition of Ad5 on porous media compared with R_{f} values for monolayer adsorption of these analytes. For 1.6 pM Ad5 this would be at the level of noise that is shown in FIG. 10. ResourceQ™ chromatography media has 100-nanometer pores that exclude Ad5 capsid from intraparticle adsorption, limiting static capacity for Ad5 to 0.14 mg/ml [5]. This value of static capacity corresponds to monolayer Ad5 adsorption on the exterior surface of the resin sphere. If ResourceQ™ were considered a homogeneous anisotropic porous media, static capacity (or Ad5 concentration of 1.6 pM) would need to increase >500-fold to distinguish an SPR response from Ad5 adsorption.

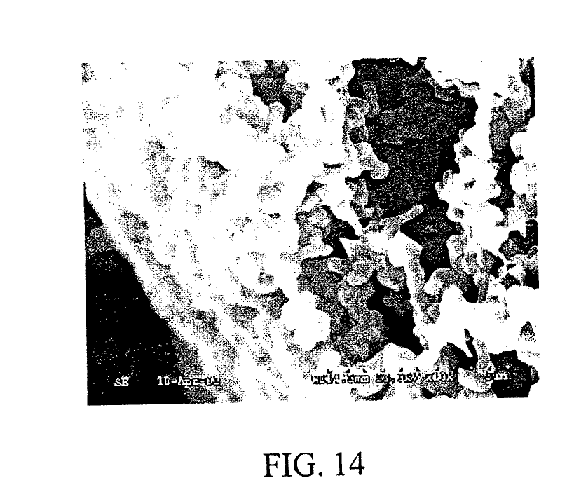

Third, analyte deposition onto monodisperse 10-μm nonporous spheres hexagonally close packed onto the sensor surface from a uniform-concentration boundary layer without adsorption onto the sensor surface will be considered. Equations 13 and 19 show the SPR response from analyte deposition is proportional to 10⁶/Qₘₐₓ and (1−ε)ζf(t)[2+ζ](ηₐ−ηₛ). The dimensionless analyte-to-adsorbent ratio, ζ, is 0.008 for Ad5 and 0.00062 for cytochrome c. Substitution yields R_{f} values of about 3.4×10⁸ RU per gram per cm² for both cytochrome c and adenovirus. These values are about 2% of comparable results for monolayer adsorption on a planar sensor. No distinguishable SPR response would be expected from 1.6 pM Ad5 adsorption on 10-μm ResourceQ™ beads. This result is consistent with the fact that a value of f(t)=0.546 has been used and that a calculation shows surface area on 10-μm spheres available for sorption within the active sensor region is ˜4% that of the underlying sensor surface. Replacing the 10-μm resin with a membrane formed by sintering polymer beads of 1-μm length scale, as illustrated in FIG. 14, would increase these R_{f} values by a factor of about 10 and raise the SPR response for 1.6 pM Ad5 to a distinguishable level.

All references, including publications, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within the known or customary practice in the art to which this invention pertains and which fall within the limits of the appended claims.

### REFERENCES

[1] Gene Therapy Clinical Trials Worldwide. Journal of Gene Medicine. http://217.215.32.12/trials/index.html
[2] W. C. Russell. Journal of General Virology. 81 (2000) 2573-2604.
[3] B. A. Rubin and L. B. Rorke, in S. A. Plotkin and E. A. Mortimer (Editors), Vaccines. 2nd Ed. Philadelphia, Pa.: W B Saunders Co., 1994 pp. 475-502.
[4] CBER, in Guidance for Human Somatic Cell Therapy and Gene Therapy, 1998.
[5] P. W. Shabram, D. D. Giroux, A. M. Goudreau, R. J. Gregory, M. T. Horn, B. G. Huyghe, X. Liu, M. H. Nunnally, B. J. Sugarman, S. Sutjipto. Human Gene Therapy (1997 Mar. 1), 8(4), 453-65.
[6] L. G. Faegerstam, A. Frostell-Karlsson, R. Karlsson, B. Persson, I. Roennberg. J. Chromatogr. (1992), 597(1-2), 397-410.
[7] Surface Plasmon Resonance. Biacore Inc. http://www.biacore.com/technology/spr_technology.lasso
[8] H. Lortat-Jacob, E. Chouin, S. Cusack and M. J. van Raaij. J Biol Chem 276:12 (2001) 9009-9015.
[9] I. Kirby, E. Davison, A. J. Beavil, C. P. C. Soh, T. J. Wickham, P. W. Roelvink, I. Kovesdi, B. J. Sutton aand G. Santis. J Virol 74:6 (March 2000) 2804-2813.
[10] P. R. Morrill, R. B. Millington, C. R. Lowe. J. Chromatogr. B. (2003), 793(2), 229-251.
[11] D. G. Myszka, X. He, M. Dembo, T. A. Morton, B. Goldstein. Biophysical Journal (1998), 75(2), 583-594.
[12] R. Cope. Nomadics, Inc. Personal Communication.
[13] Jung, Linda S.; Campbell, Charles T.; Chinowsky, Timothy M.; Mar, Mimi N.; Yee, Sinclair S. Langmuir (1998), 14(19), 5636-5648.
[14] Chinowsky, T. M.; Quinn, J. G.; Bartholomew, D. U.; Kaiser, R.; Elkind, J. L. Sensors and Actuators, B: Chemical (2003), B91(1-3), 266-274.
[15] Zhang, Liqin; Vidu, Ruxandra; Waring, Alan J.; Lehrer, Robert I.; Longo, Marjorie L.; Stroeve, Pieter. Langmuir (2002), 18(4), 1318-1331.
[16] E. Margoliash and A. Schejter, in R. A. Scott and A. G. Mauk (Editors), Cytochrome C. A Multidisciplinary Approach. University Science Books: Sausalito, Calif. (1996) pp. 3-32.
[17] M. Beissinger, H. Sticht, M. Sutter, A. Ejchart, W. Haehnel, P. Rosch, EMBO J (1998) 17 pp. 27.
[18] E. Hallgren. F. Kalman, D. Farnan, C. Horvath, J. Stahlberg. J. Chromatogr., A (2000), 877(1+2), 13-24.
[19] R. K. Scopes. Protein Purification. Principles and Practice. Springer Verlag: New York 1987.
[20] W. Leyko, R. Gondko. Biochimica et Biophysica Acta (1963), 77(3), 500-1.
[21] N. Z. Shengelidze, B. I. Chumburidze, M. G. Chirikashvili. Khromatogr. Metody Farm. (1977), 79-84.
[22] Z. Qi, N. Matsuda, A. Takatsu, K. Kato. J. Phys. Chem. B (2003), 107(28), 6873-6875.
[23] J. S. Salafsky and K. B. Eisenthal. J. Phys. Chem B. (2000) 104(32), 7752-7755.
[24] A. C. Allison and R. C. Valentine. Biochem Biophys Acta (1960) 40:393-399.
[25] A. Garnier, P.-A. Gilbert and A. Kamen. Computer Applications in Biotechnology 2001: Modelling, Monitoring and Control of Biotechnical Processes, A Proceedings Volume from the IFAC International Conference, 8th, Quebec City, QC, Canada, Jun. 24-27, 2001 (2002).
[26] R. Galinier, E. Gout, H. Lortat-Jacob, J. Wood, J. Chroboczek. Biochemistry (2002), 41(48), 14299-14305.
[27] Leveque, M. Ann. Mines (1928) 13:284.
[28] Koch, D. L. Journal of Fluid Mechanics (1996) 318:31-47.
[29] Gunn, D. J. Chemical Engineering Science (1987) 42(2):363-73.
[30] Moutsopoulos, K. N., and D. L. Koch. Journal of Fluid Mechanics (1999) 385:359-379.
[31] Edelstein, M. (2004) Gene Therapy Clinical Trials Worldwide. (2004) The Journal of Gene Medicine http://www.wiley.co.uk/genetherapy/clinical/ Accessed 8/04.
[32] Russell, W. C. Journal of General Virology (2000) 81:2573-2604.
[33] Margoliash, E., and A. Schejter. (1996)In Cytochrome C, Scott, R. A.; Mauk, A. G. Eds; University Science Books: Sausalito, Calif. 3-32.
[34] Roper, D. K. and G. Purdom. (2004) Adenovirus Binding, Elution and Equilibrium Measured by Surface Plasmon Resonance. PREP 2004, Baltimore, Md. May 24-26
[35] Seader, J. D. and E. J. Henley. (1998) Separation Process Principles. John Wiley and Sons. Hoboken, N.J.
[36] Wofsy, C and B. Goldstein. Biophysical Journal (2002) 82(4):1743-1755.
[37] Jung, L. S., C. T. Campbell, T. M. Chinowsky, M. N. Mar, and S. S. Yee. Langmuir (1998) 14(19):5636-5648.
[38] Davies, J., and I. Faulker. (1996) Surface Plasmon Resonance—Theory and Experimental Consideration. In Chemistry and Physics of Surfaces and Interface. Surface Analytical Techniques for Probing Biomaterial Processes. J. Davis, editor. CRC Press: Boca Raton, Fla. 67-87.
[39] Moghimi, S. M., K. D. Pavey, and A. C. Hunter. FEBS letters (2003) 547(1-3):177-82.
[40] Lok, B. K., Y. L. Cheng, and C. R. Robertson. Journal of Colloid and Interface Science (1983) 91(1):87-103.
[41] Ramsden, J. J., S.-Y. Li, J. E. Prenosil, and E. Heinzle. Biotechnol. Bioeng. (1994) 43:939-45

What is claimed is:

1. A method for detecting or analyzing binding interactions analyzing between an analyte and a three-dimensional surface, said method comprising:
providing a surface plasmon resonance sensor chip;
further providing a three-dimensional surface in the sensing region of said surface plasmon resonance sensor chip;
exposing said sensor chip to the analyte; and
transforming the change in refractive index so as to determine the magnitude of a binding interaction forward rate constant and reverse rate constant.

2. The method according to claim 1 further comprising detecting the binding interaction of the analyte with the 3-dimensional surface by measuring a change in refractive index.

3. The method according to claim 1, wherein the three-dimensional surface is a self-assembled monolayer formed on a surface plasmon resonance sensor chip.

4. The method according to claim 2, wherein the self-assembled monolayer on a surface plasmon resonance sensor chip is formed by process comprising derivatizing a sulfhydryl group of 11-mercaptoundecanoic acid with N,N-diethylethylenediamine.

5. The method according to claim 1, wherein the analyte comprises adenovirus.

6. The method according to claim 1, wherein the analyte comprises cytochrome c.

7. The method according to claim 1, wherein the analyte comprises a biomolecule with a mass greater than or equal to about 10⁶ Da.

8. The method according to claim 1, wherein exposing said sensor chip to the analyte comprises incorporating porous media so as to increase the lateral mass transport through the process of hydrodynamic diffusion or radial dispersion.

9. The method according to claim 1, wherein transforming the change in refractive index so as to determine the magnitude of a binding interaction forward rate constant and reverse rate constant comprises a transformation based on uniform adsorption of homogeneous analyte solutions on homogeneous adsorptive sites distributed heterogeneously in space relative to a planar boundary.

10. A method for detecting or analyzing binding interactions between an analyte and a self-assembled monolayer, said method comprising:
immobilizing a self-assembled monolayer on a surface plasmon resonance sensor chip;
exposing said sensor chip to the analyte; and
transforming the change in refractive index so as to determine the magnitude of a binding interaction forward rate constant and reverse rate constant.

11. The method according to claim 10 further comprising detecting the binding interaction of the analyte with the monolayer by measuring a change in refractive index.

12. The method according to claim 10, wherein forming a self-assembled monolayer on a surface plasmon resonance sensor chip comprises derivatizing a sulfhydryl group of 11-mercaptoundecanoic acid with N,N-diethylethylenediamine.

13. The method according to claim 10, wherein the analyte comprises adenovirus.

14. The method according to claim 10, wherein the analyte comprises cytochrome c.

15. The method according to claim 10, wherein the analyte comprises a biomolecule with a mass greater than or equal to about 10⁶ Da.

16. The method according to claim 10, wherein exposing said sensor chip to the analyte comprises incorporating porous media so as to increase the lateral mass transport through the process of hydrodynamic diffusion or radial dispersion.

17. The method according to claim 10, wherein transforming the change in refractive index so as to determine the magnitude of a binding interaction forward rate constant and reverse rate constant comprises a transformation based on uniform adsorption of homogeneous analyte solutions on homogeneous adsorptive sites distributed heterogeneously in space relative to a planar boundary.

18. A method for detecting or analyzing binding interactions between an analyte and a self-assembled monolayer, said method comprising:
providing a surface plasmon resonance sensor chip;
exposing said sensor chip to the analyte;
incorporating porous media, so as to increase the lateral mass transport through hydrodynamic diffusion; and
detecting the interaction of the analyte with the monolayer by measuring a change in refractive index.

19. A method of characterizing the interaction between an analyte and three-dimensional surface using surface plasmon resonance, wherein lateral mass transport is increased by incorporation of porous media in a flow cell of a surface plasmon resonance detection device.

20. The method according to claim 19, wherein the porous media comprises a fibrous bed or concentrated bed of spheres.

21. An apparatus for detecting or analyzing binding interactions on a three-dimensional surface, said apparatus comprising:
a surface plasmon resonance sensor chip; and
a three-dimensional surface in the sensing region of said surface plasmon resonance sensor chip

22. The apparatus according to claim 21, wherein the three-dimensional surface comprises a fibrous bed or bed of spheres.

23. The apparatus according to claim 21, wherein the three-dimensional surface is a self-assembled monolayer.
